(19) United States
(12) Patent Application Publication
(10) Pub. No.: US 2025/0285233 A1
(43) Pub. Date: Sep. 11, 2025

(54) INFORMATION PROCESSING SYSTEM, ENDOSCOPE SYSTEM, IMAGE PROCESSING METHOD AND INFORMATION STORAGE MEDIUM

(71) Applicant: OLYMPUS MEDICAL SYSTEMS CORP., Tokyo (JP)

(72) Inventors: Shota NAKAJIMA, Tokyo (JP); Keigo MATSUO, Tokyo (JP); Tetsuhiro OKA, Tokyo (JP)

(73) Assignee: OLYMPUS MEDICAL SYSTEMS CORP., Tokyo (JP)

(21) Appl. No.: 19/049,316

(22) Filed: Feb. 10, 2025

(30) Foreign Application Priority Data
Mar. 7, 2024 (JP) 2024-035184

(51) Int. Cl. G06T 5/60; G06T 5/20; G06T 5/73; G06T 7/50; G16H 30/40

(52) U.S. Cl. CPC G06T 5/60; G06T 5/20; G06T 5/73; G06T 7/50; G16H 30/40; G06T 2207/10068; G06T 2207/20021; G06T 2207/20048; G06T 2207/20081; G06T 2207/20084; G06T 2207/30004

(57) ABSTRACT

Defocus simulation processing is performed for a region on an optical axis of a first imaging system and a region other than on the optical axis in a training image, based on a transfer function or a point spread function on the optical axis. One or more processors use a trained model to generate an output image in which a blur of a processing target image which is an image captured by the first imaging system is corrected, and estimates an object distance of the processing target image. The one or more processors acquire a filter characteristic associated with the estimated object distance from a correction table, and performs blur adjustment processing for the output image using the acquired filter characteristic.

### CROSS REFERENCE TO RELATED APPLICATIONS

This application is based upon and claims the benefit of priority to Japanese Patent Application No. 2024-035184 filed on Mar. 7, 2024, the entire contents of which are incorporated herein by reference.

### BACKGROUND

In endoscopic observation, for example, magnifying observation closer to a subject is desired. Optically, however, a depth of field is narrower with a higher resolution with finer pixels. Thus, technologies for extending the depth of field using image processing techniques have been sought. WO 2018/037521 discloses a technology that corrects optical degradation of an imaging system by deep learning.

WO 2018/037521 uses, as a training image, a reference image captured in advance with optical degradation information. However, there are myriads of optical degradation information to learn depending on object distance and image height, and therefore an enormous number of training images are required. Consequently, the network scale necessary for processing increases, leading to reduction in processing ability and increase in implementation costs.

### SUMMARY

In accordance with one of some aspect, there is provided an information processing system comprising:
a memory configured to store a trained model trained by machine learning with a data set including a training image, a true image, and an object distance label, and a correction table in which an object distance is associated with a filter characteristic for blur correction, the object distance being a distance between an imaging system and a subject; and
one or more processors, wherein
the training image is generated by performing defocus simulation processing that simulates, for a predetermined subject image in focus captured by a given imaging system, an effect of a blur caused by defocus of a first imaging system, based on a transfer function or a point spread function of the first imaging system at a predetermined object distance,
the defocus simulation processing is performed for a region on an optical axis of the first imaging system and a region other than on the optical axis in the training image, based on the transfer function or the point spread function on the optical axis,
the true image is an image generated by performing best focus simulation processing that simulates, for the predetermined subject image, a state in which the first imaging system is focused, based on the transfer function or the point spread function at the object distance at which the first imaging system is focused, or the predetermined subject image itself,
the trained model is trained by machine learning so that the training image is the true image, and trained by machine learning by applying, as the object distance label, the object distance of the transfer function or the point spread function of the first imaging system used in the defocus simulation processing, and
the one or more processors
   use the trained model to generate an output image in which a blur of a processing target image is corrected, the processing target image being an image captured by the first imaging system,
   estimate the object distance of the processing target image, and
   acquire, from the correction table, the filter characteristic associated with the estimated object distance, and perform blur adjustment processing for the output image using the acquired filter characteristic.

In accordance with one of some aspect, there is provided an endoscope system comprising:
the information processing system according to any one of claims 1 to 9; and
an endoscopic scope configured to capture the processing target image.

In accordance with one of some aspect, there is provided an image processing method using a trained model trained by machine learning with a data set including a training image, a true image, and an object distance label, and a correction table in which an object distance is associated with a filter characteristic for blur correction, the object distance being a distance between an imaging system and a subject, wherein
the training image is generated by performing defocus simulation processing that simulates, for a predetermined subject image in focus captured by a given imaging system, an effect of a blur caused by defocus of a first imaging system, based on a transfer function or a point spread function of the first imaging system at a predetermined object distance,
the defocus simulation processing is performed for a region on an optical axis of the first imaging system and a region other than on the optical axis in the training image, based on the transfer function or the point spread function on the optical axis,
the true image is an image generated by performing best focus simulation processing that simulates, for the predetermined subject image, a state in which the first imaging system is focused, based on the transfer function or the point spread function at the object distance at which the first imaging system is focused, or the predetermined subject image itself,
the trained model is trained by machine learning so that the training image is the true image, and trained by machine learning by applying, as the object distance label, the object distance of the transfer function or the point spread function of the first imaging system used in the defocus simulation processing,
the image processing method comprising:
using the trained model to generate an output image in which a blur of a processing target image is corrected, the processing target image being an image captured by the first imaging system;
estimating the object distance of the processing target image; and
acquiring, from the correction table, the filter characteristic associated with the estimated object distance, and performing blur adjustment processing for the output image using the acquired filter characteristic.

In accordance with one of some aspect, there is provided a non-transitory information storage medium that stores a program for causing a computer to execute the image processing method according to claim 11.

### BRIEF DESCRIPTION OF THE DRAWINGS

FIG. 1 is a block diagram illustrating a configuration example of an information processing system.

FIG. 2 is a block diagram illustrating the configuration example of the information processing system in more detail.

FIG. 3 is a flowchart illustrating a processing example of the information processing system.

FIG. 4 is a block diagram illustrating a configuration example of a training device.

FIG. 5 is a diagram illustrating a training model.

FIG. 6 is a diagram illustrating a neural network.

FIG. 7 is a flowchart illustrating trained model creation processing.

FIG. 8 is a diagram illustrating machine learning according to an embodiment of the present disclosure.

FIG. 9 is a diagram illustrating a relationship between a depth of field and a target depth of field.

FIG. 10 is a diagram illustrating image data generation processing according to an embodiment.

FIG. 11 is a diagram illustrating the image data generation processing according to another embodiment.

FIG. 12 is a diagram illustrating a transfer function or a point spread function.

FIG. 13 is a diagram illustrating defocus simulation processing according to an embodiment of the present disclosure.

FIG. 14 is a block diagram illustrating an endoscope system according to an embodiment.

FIG. 15 is a block diagram illustrating the endoscope system according to another embodiment.

FIG. 16 is a graph illustrating a relationship between an object distance and MTF in the defocus simulation processing.

FIG. 17 is another graph illustrating a relationship between an object distance and MTF in the defocus simulation processing.

FIG. 18 is a diagram illustrating a specific computation technique in the defocus simulation processing.

FIG. 19 is another diagram illustrating a specific computation technique in the defocus simulation processing.

FIG. 20 is a diagram illustrating a specific computation technique in best focus simulation processing.

FIG. 21 is another diagram illustrating a specific computation technique in the best focus simulation processing.

FIG. 22 is a diagram illustrating a lens configuration of a first imaging system according to an embodiment.

FIG. 23 is a block diagram illustrating another configuration example of the information processing system.

FIG. 24 is a block diagram illustrating another configuration example of the information processing system in more detail.

FIG. 25 is a flowchart illustrating another processing example of the information processing system.

FIG. 26 is a graph illustrating a relationship between an object distance and MTF in the defocus simulation processing.

FIG. 27 is another graph illustrating a relationship between an object distance and MTF in the defocus simulation processing.

FIG. 28 is a diagram illustrating a specific computation technique in the defocus simulation processing.

FIG. 29 is another diagram illustrating a specific computation technique in the defocus simulation processing.

FIG. 30 is a diagram illustrating a specific computation technique in best focus simulation processing.

FIG. 31 is another diagram illustrating a specific computation technique in the best focus simulation processing.

FIG. 32 is a diagram illustrating a lens configuration of a first imaging system according to an embodiment.

FIG. 33 is another diagram illustrating a lens configuration of the first imaging system according to another embodiment.

FIG. 34 is a diagram illustrating an amount of distortion.

FIG. 35 is a diagram illustrating a lens configuration including a phase modulation element.

FIG. 36 is a graph illustrating change in MTF due to inclusion of the phase modulation element according to an embodiment.

FIG. 37 is a diagram illustrating the image data generation processing according to another embodiment.

FIG. 38 is a diagram illustrating the defocus simulation processing according to another embodiment.

FIG. 39 is a diagram illustrating the image data generation processing according to another embodiment.

FIG. 40 is a diagram illustrating the defocus simulation processing according to another embodiment.

FIG. 41 is a diagram illustrating the best focus simulation processing according to another embodiment.

FIG. 42 is a diagram illustrating another configuration example of the information processing system.

FIG. 43 is a diagram illustrating the image data generation processing according to another embodiment.

FIG. 44 is a diagram illustrating the defocus simulation processing according to another embodiment.

FIG. 45 is a diagram illustrating a relationship between mosaicing processing and demosaicing processing.

FIG. 46 is a diagram illustrating the best focus simulation processing according to another embodiment.

FIG. 47 is a diagram illustrating another configuration example of the information processing system.

FIG. 48 is a flowchart illustrating another processing example of the information processing system.

FIG. 49 is a flowchart illustrating first trained model creation processing.

FIG. 50 is a flowchart illustrating second trained model creation processing.

FIG. 51 is a diagram illustrating the image data generation processing according to another embodiment.

FIG. 52 is a diagram illustrating the defocus simulation processing according to another embodiment.

FIG. 53 is a diagram illustrating the best focus simulation processing according to another embodiment.

FIG. 54 is a diagram illustrating evaluation of a frequency characteristic.

FIG. 55 is a diagram illustrating the relationship between a first frequency characteristic and a second frequency characteristic, and the like.

FIG. 56 is a diagram illustrating a modification of correction processing.

FIG. 57 is a diagram illustrating adjustment of a blur for an output image in more detail.

FIG. 58 is another diagram illustrating adjustment of a blur for an output image in more detail.

FIG. 59 is a diagram illustrating another configuration example of the trained model.

### DESCRIPTION OF EXEMPLARY EMBODIMENTS

The following disclosure provides many different embodiments or examples for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

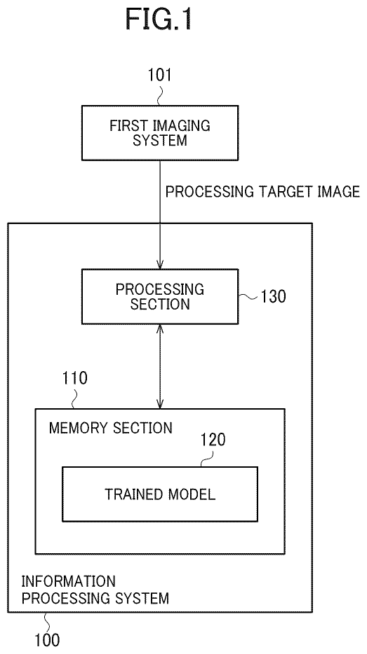

FIG. 1 is a block diagram illustrating a configuration example of an information processing system 100 according to an embodiment of the present disclosure. The present information processing system 100 includes a memory section 110 and a processing section 130. The memory section 110 stores a trained model 120 trained by machine learning. The trained model 120 is a program module that outputs a corrected image in which a blur caused by defocus in a processing target image is corrected. The trained model 120 is generated or updated by performing machine learning described later. The processing target image is, for example, image data captured by a first imaging system 101 as illustrated in FIG. 1, but not limited thereto. The details will be described later. In the present embodiment, image data that can be processed as digital data may be simply referred to as image. A training image group 32G is a set of training images 32 including a first training image 32-1, a second training image 32-2, . . . , and an Nth training image 32-N. The training image group 32G will be detailed later in conjunction with a true image 36. In other words, the processing section 130 in the present embodiment uses the trained model 120 to correct a blur caused by defocus of the first imaging system 101 in the processing target image which is an image captured by the first imaging system 101. The memory section 110 and the processing section 130 are also referred to as a memory device and a processing device, respectively.

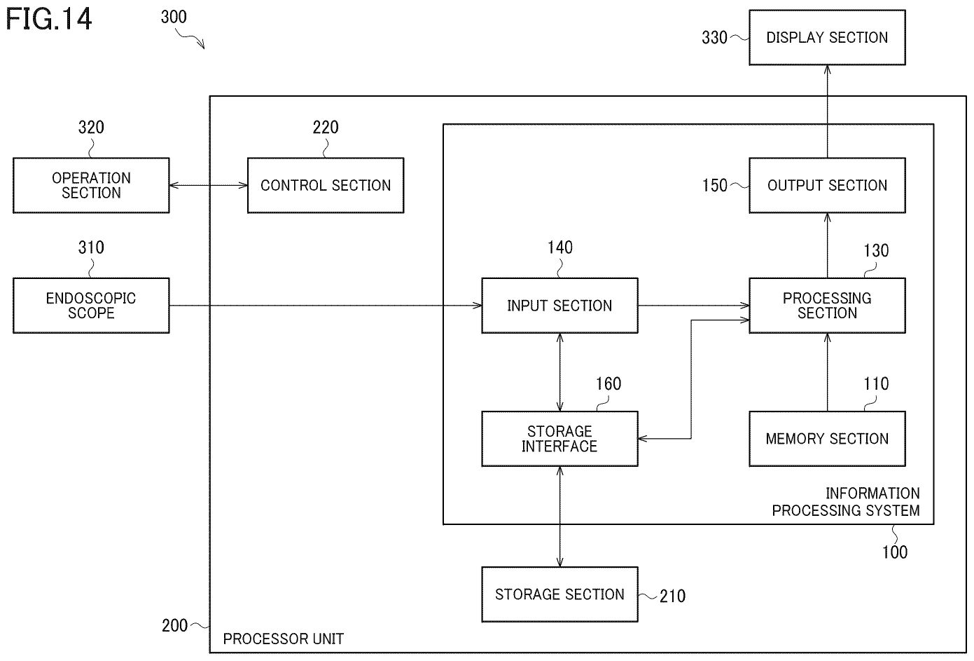

When the information processing system 100 is incorporated into an endoscope system 300 described later with reference to FIG. 14 and the like, the training image 32 in the present embodiment is, for example, but not limited to, an image of biological tissue or an image of a subject that imitates biological tissue. For example, in a learning phase described later, an image of a chart that conforms to a predetermined standard, such as a spatial frequency response (SFR) chart, may be employed as the training image 32. An image of a chart with a predetermined modification to such a chart image may also be employed as the training image 32. The predetermined modification is, for example, a modification that makes the contrast of the chart common to the contrast of biological tissue.

Machine learning in the present embodiment is, for example, supervised learning. Training data in supervised learning is a data set in which input data is associated with a ground truth label. Specifically, the trained model 120 in the present embodiment is generated by supervised learning based on a data set in which input data including the training images 32 simulating the effects of various kinds of blur is associated with a ground truth label including the true image 36 in focus.

The processing section 130 in the present embodiment is configured with hardware described below. The hardware can include at least one of a circuit that processes digital signals and a circuit that processes analog signals. For example, the hardware can be configured with one or more circuit devices or one or more circuit elements mounted on a circuit board. One or more circuit devices are, for example, ICs. One or more circuit elements are, for example, capacitors.

The processing section 130 may be implemented, for example, by a processor described below. The processing section 130 in the present embodiment includes a memory that stores information and a processor that operates based on the information stored in the memory. The memory is, for example, the memory section 110. The information is, for example, a program and various data. The processor includes hardware. Various processors such as a central processing unit (CPU), a graphics processing unit (GPU), and a digital signal processor (DSP) can be used as the processor. The memory may be a semiconductor memory such as a static random access memory (SRAM) or a dynamic random access memory (DRAM), a register, a magnetic storage device such as a hard disc device, or an optical storage device such as an optical disc device. For example, the memory stores computer-readable instructions, and the instructions are executed by the processor to implement the function of each unit of the processing section 130 as processing. As used herein the instructions may be instructions of an instruction set that constitutes a program or may be instructions to instruct the hardware circuit of the processor to operate.

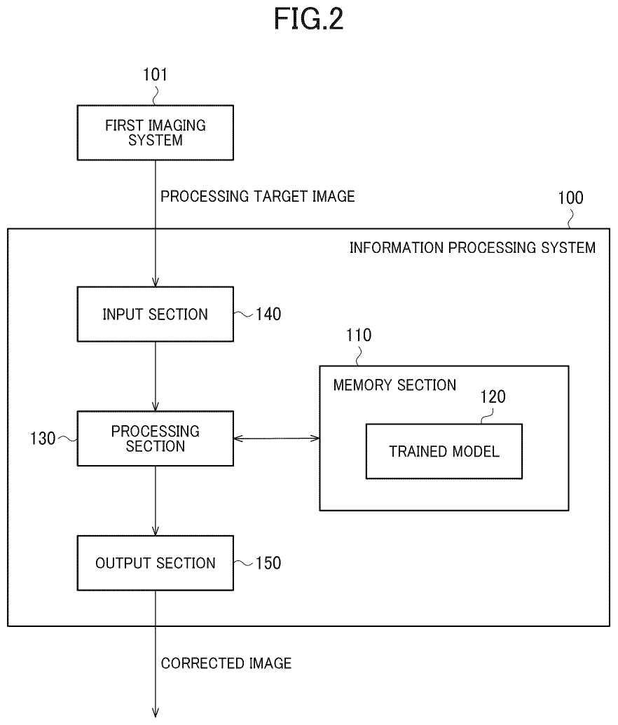

Further, the trained model 120 in the present embodiment may be used by the information processing system 100 depicted in a configuration example in FIG. 2. In other words, the trained model 120 in the present embodiment is used by the information processing system 100 including the memory section 110 that stores the trained model 120, an input section 140, the processing section 130, and an output section 150, and is trained by machine learning using a data set including the training image group 32G and the true image 36.

The input section 140 is an interface that receives the processing target image from outside. Specifically, for example, as illustrated in FIG. 1 and FIG. 2, the input section 140 is an image data interface that receives image data as the processing target image from the first imaging system 101. For example, the input section 140 inputs the received processing target image as input data to the trained model 120 and the processing section 130 performs processing described later, whereby the function as the input section 140 is served. In other words, in the trained model 120 in the present embodiment, the input section 140 inputs the processing target image, which is an image captured by the first imaging system 101, to the trained model 120.

The output section 150 is an interface that transmits the corrected image described above to the outside. For example, output data from the trained model 120 is used as the corrected image transmitted by the output section 150, whereby the function as the output section 150 is served. The corrected image is transmitted to, for example, a predetermined display device connected to the information processing system 100. For example, the output section 150 is an interface that can be connected to the predetermined display device so that the corrected image appears on the display device, whereby the function as the output section 150 is served. The corrected image may be output to, for example, a storage device which is an external device.

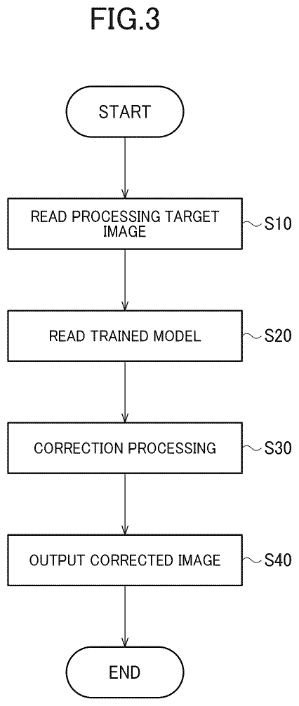

FIG. 3 is a flowchart illustrating a technique performed by the information processing system 100 according to the present embodiment. The processing section 130 reads the processing target image (step S10) and reads the trained model (step S20), and thereafter performs correction processing (step S30). Specifically, for example, the processing section 130 performs processing of inputting the processing target image received via the input section 140 to the trained model 120 read from the memory section 110. If it is determined that the processing target image, which is input data, is common to the training image 32, the trained model 120 estimates that data to be output is the true image 36. Thus, upon input of the processing target image, the trained model 120 outputs the true image 36. When the processing target image and the true image 36 are compared, there is a relationship such that the true image 36 is an image in which a blur caused by defocus of the first imaging system 101 in the processing target image is corrected. In other words, the processing section 130 performs the correction processing of correcting a blur caused by defocus of the first imaging system 101 in the processing target image, using the trained model 120 (step S30).

The processing section 130 thereafter outputs the corrected image (step S40). Specifically, the output section 150 functions as described above whereby the corrected image is output to a desired destination. In other words, the output section 150 outputs the corrected image produced by the correction processing.

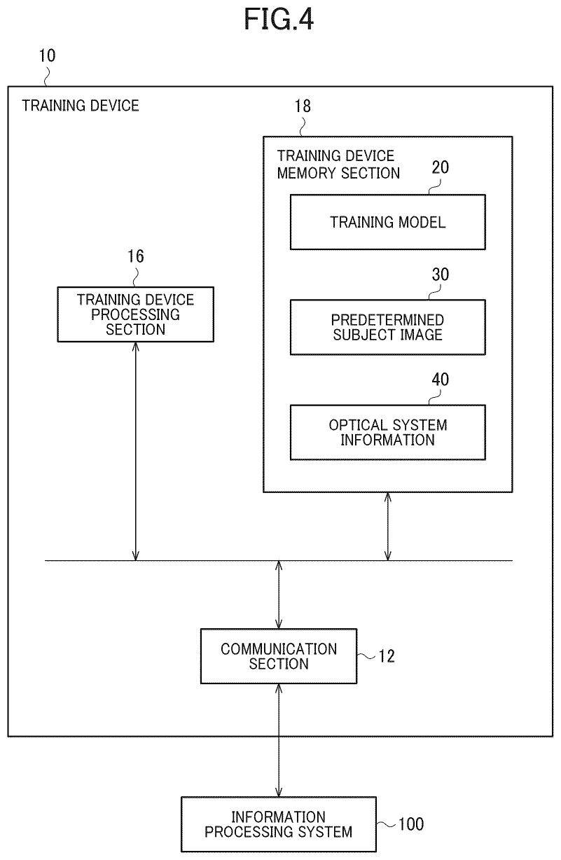

The machine learning of the trained model 120 will now be described. The machine learning is performed, for example, by a training device 10. FIG. 4 is a block diagram illustrating a configuration example of the training device 10. The training device 10 includes, for example, a communication section 12, a training device processing section 16, and a training device memory section 18.

The communication section 12 is a communication interface that can communicate with the information processing system 100 via a predetermined communication scheme. The predetermined communication scheme is, for example, a communication scheme in conformity with a wireless communication standard such as Wi-Fi (registered trademark), but not limited thereto, and may be a communication scheme in conformity with a wired communication standard such as USB. With this configuration, the training device 10 can transmit the trained model 120 trained by machine learning according to a technique described later to the information processing system 100, and the information processing system 100 can update the trained model 120. Although FIG. 4 illustrates an example in which the training device 10 is separate from the information processing system 100, it is not intended to preclude a configuration example in which the information processing system 100 includes a training server corresponding to the training device 10.

The training device processing section 16 performs input/output control of data to/from each functional unit such as the communication section 12 and the training device memory section 18. The training device processing section 16 can be implemented by a processor similar to the processing section 130 in FIG. 1. The training device processing section 16 controls operation such as data output to the information processing system 100 by executing various computation processing based on a predetermined program read from the training device memory section 18 and an operation input signal and the like from an operation unit not illustrated in FIG. 4. The predetermined program here includes a machine learning program. In other words, the training device processing section 16 serves the function of machine learning by reading the machine learning program, necessary data, and the like from the training device memory section 18 and executing the machine learning program.

The training device memory section 18 stores a training model 20, a predetermined subject image 30, and optical system information 40, in addition to the not-illustrated machine learning program. The training device memory section 18 can be implemented by, for example, a semiconductor memory similar to the memory section 110 described above. The training device memory section 18 may further include another information. Another information is, for example, image sensor information 50 described later.

The predetermined subject image 30 is an image of a subject associated with the processing target image. The training image 32 and the true image 36, which will be described later, are created based on the predetermined subject image 30. In other words, the training device memory section 18 stores predetermined subject images 30 as many as the kinds of subjects from which processing target images can be produced. To give a more specific example, in a case where the information processing system 100 is used for an endoscope system 300 described later, a captured image of a lumen or the like captured by an endoscopic scope 310 described later is the predetermined subject image 30. In the following description, an imaging system that captures the predetermined subject image 30 is referred to as a given imaging system 104, unless otherwise specified. A case where the predetermined subject image 30 is captured with a specified imaging system will be described later.

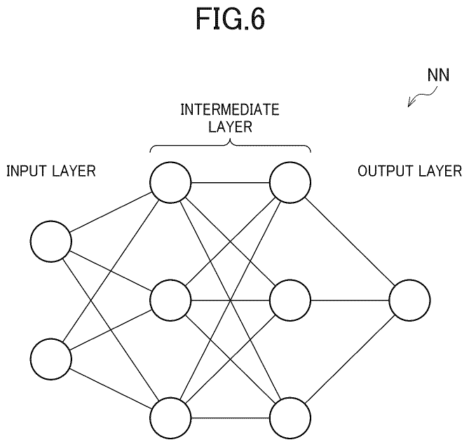

The training model 20 is a model subjected to machine learning by the training device processing section 16. The model here is information that derives a correspondence between estimation target data and estimation result data. More specifically, the model is information that derives an output image 34 which is the estimation result data from the training image 32 which is the estimation target data. In the training model 20 in the present embodiment, a neural network NN is included in at least a part of the model. The detail of the neural network NN will be described later with reference to FIG. 6. In the case where the information processing system 100 and the training device 10 are integrated as described above, the trained model 120 may be subjected to machine learning.

The “output image” in the present embodiment refers to an image output from the training model 20 in the learning phase, or an image output from a training model 1020 described later, but may also include an image output from a trained model 1120 in an inference phase in an example described later. Hereinafter, for convenience of explanation, an image output from the training model 20 or the training model 1020 is referred to as output image 34, and an image output from the trained model 1120 described later is referred to as output image 134.

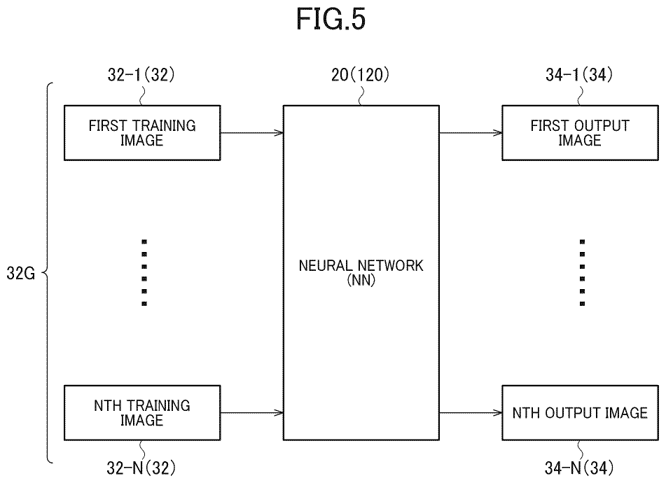

For example, when the first training image 32-1 is input to the training model 20, the training model 20 outputs a first output image 34-1. Similarly, when the Nth training image 32-N is input to the training model 20, the training model 20 outputs an Nth output image 34-N. In other words, as illustrated in FIG. 5, in the training device 10 according to the present embodiment, N images including the first training image 32-1 to the Nth training image 32-N are input as the training image group 32G to the training model 20.

FIG. 6 is a diagram illustrating the neural network NN. The neural network NN includes an input layer that receives data, an intermediate layer that performs computation based on an output from the input layer, and an output layer that outputs data based on an output from the intermediate layer. FIG. 6 illustrates a network including two intermediate layers by way of example, but the network may include one intermediate layer or three or more intermediate layers. The number of nodes included in each layer is not limited to the number in the example in FIG. 6, and a variety of modifications may be implemented. As illustrated in FIG. 6, nodes included in a given layer are connected to nodes in an adjacent layer. A weighting factor is set for each connection. Each node multiplies an output from a node in the preceding layer by the weighting factor and obtains a sum of multiplication results. In addition, each node adds a bias to the sum and applies an activation function to the addition result to obtain an output of the node. This processing is successively executed from the input layer toward the output layer, resulting in an output of the neural network NN. Various functions such as sigmoid and ReLU functions are known as the activation function and can be widely applied in the present embodiment.

Models with various configurations are known for the neural network NN and can be widely applied in the present embodiment. For example, the neural network NN may be a convolutional neural network (CNN), a recurrent neural network (RNN), or other models.

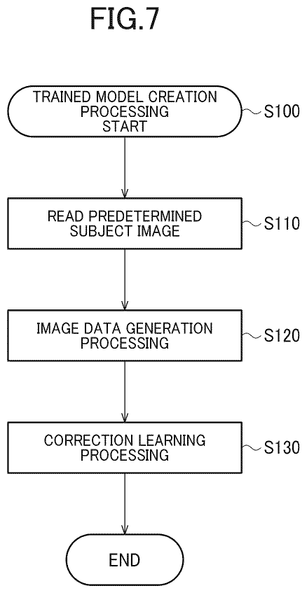

FIG. 7 is a flowchart illustrating a processing example of trained model creation processing (step S100). The trained model creation processing (step S100) is processing of creating or updating the trained model 120 by machine learning. The training device processing section 16 reads the predetermined subject image (step S110) and thereafter performs image data generation processing (step S120). For example, the training device processing section 16 reads the predetermined subject image 30 from the training device memory section 18 and performs predetermined processing for generating the training image 32 and the true image 36 using the predetermined subject image 30. The predetermined processing is, for example, defocus simulation processing (step S200) and best focus simulation processing (step S300), which will be detailed later.

The training device processing section 16 thereafter performs correction learning processing (step S130). For example, the training device processing section 16 performs processing of reading the training model 20 from the training device memory section 18, processing of inputting the training image 32 generated in the image data generation processing (step S120) to the training model 20, and machine learning processing based on the output image 34 output from the training model 20 and the true image 36.

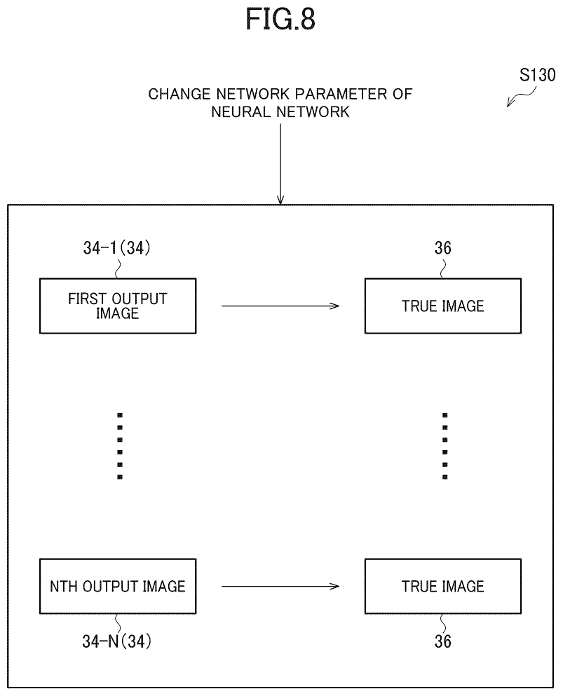

The machine learning processing based on the output image 34 and the true image 36 is processing of changing a network parameter of the neural network NN so that the first output image 34-1 to Nth output image 34-N are the true image 36, for example, as illustrated in FIG. 8. The processing of changing a network parameter of the neural network NN is specifically, for example, processing of updating an appropriate weighting factor in the neural network NN. The weighting factor here includes a bias. In the updating of the weighting factor, for example, back propagation can be used, which updates the weighting factor from the output layer toward the input layer. In other words, the training device 10 inputs input data of training data to a model and obtains an output by performing forward computation in accordance with a model configuration using the weighting factor at that moment. An error function is calculated based on the output and the ground truth label, and the weighting factor is updated so that the error function is reduced.

More specifically, for example, the training device processing section 16 inputs the first training image 32-1 as input data to the neural network NN included in the training model 20, and outputs the first output image 34-1 as output data by performing computation in the forward direction using the weighting factor at that moment. The training device processing section 16 computes the error function, based on the first output image 34-1 and the true image 36 which is the ground truth label. The training device processing section 16 then performs processing of updating the weighting factor so that the error function is reduced. Further, the training device processing section 16 repeatedly performs similar processing for the second output image 34-2 to the Nth output image 34-N. In this way, the training model 20 is trained by machine learning so that one true image 36 can be output for different kinds of training images 32. With this configuration, the training model 20 trained by machine learning is output as the trained model 120 to the information processing system 100, whereby the trained model 120 stored in the memory section 110 is updated. Although FIG. 4 illustrates the training device 10 and the information processing system 100 communicatively connected via the communication section 12, the training device 10 and the information processing system 100 are not necessarily communicatively connected. In this case, for example, a user performs processing on the training device 10 to temporarily store the training model 20 as the trained model 120 into an information storage medium, and brings the information storage medium to where the information processing system 100 is present, and performs processing on the information processing system 100 to update the trained model 120 based on the information storage medium, thereby implementing updating of the trained model 120.

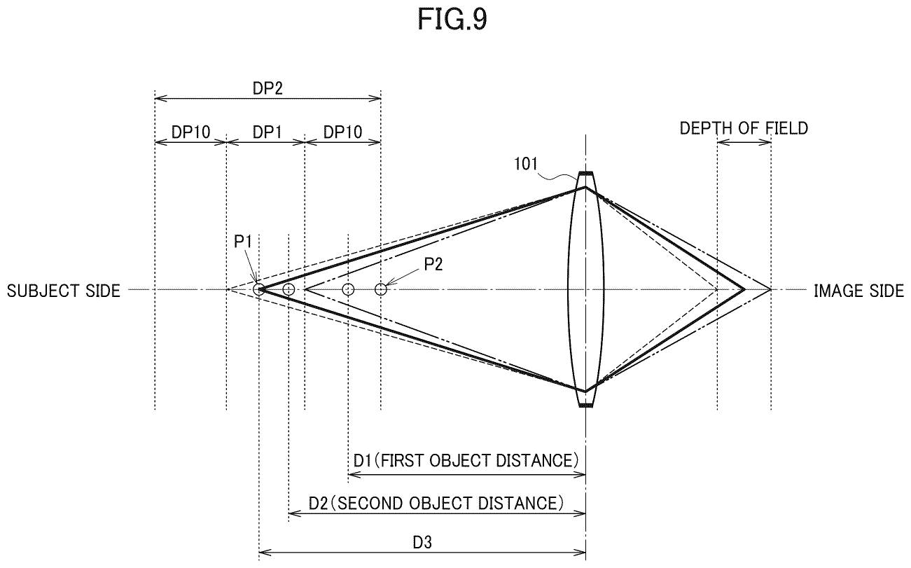

FIG. 9 is a diagram illustrating a relationship between a focal depth and a depth of field for the first imaging system 101 in the present embodiment, where the optical axis is the horizontal axis. FIG. 9 is an illustration for convenience and does not depict a specific lens configuration of the first imaging system 101. For example, in FIG. 9, the range indicated by DP1 is a depth of field corresponding to the focal depth in optical design of the first imaging system 101. Therefore, for example, when the distance between a subject and the first imaging system 101 is a first object distance indicated by D1, the subject is located outside the range of the depth of field and therefore a processing target image captured with the first imaging system 101 includes the effect of a blur caused by defocus. For example, when the distance between the subject and the first imaging system 101 is a second object distance indicated by D2, the subject is located inside the depth of field, resulting in a processing target image in focus. For example, when the distance between the subject and the first imaging system 101 is an object distance indicated by D3, that is, a position indicated by P1 on the optical axis in the depth of field is a position that satisfies a best focus condition. In FIG. 9, the first object distance indicated by D1 and the second object distance indicated by D2 are located on the near-point side relative to the position indicated by P1, but may be on the far-point side rather than the near-point side. In the following description and illustration, techniques according to the present embodiment will be described using the object distance on the near-point side and the like as an example. However, it is not intended to preclude the techniques according to the present embodiment from being applied using the object distance on the far-point side and the like.

For example, in a system including the first imaging system 101, there is a need for extending the depth of field because increasing a resolution with finer pixels narrows the depth of field. Further, for example, when the first imaging system 101 is used in the endoscopic scope 310 of the endoscope system 300 described later, the operation of adjusting the endoscopic scope 310 to a best focus position for a desired subject involves difficulty. Thus, there is a need for extending the depth of field.

Then, in the present embodiment, the information processing system 100 incorporates the trained model 120 trained by machine learning described above with reference to FIG. 8 and the like, using a data set including an image that simulates the effect of a blur for the predetermined subject image 30 captured in advance as the training image 32, and an image in focus as the true image 36. In this manner, the processing in FIG. 3 is performed for the captured image with the effect of a blur caused by defocus as the processing target image, whereby the corrected image in focus is output from the information processing system 100. As a result, the range of depth of field of the first imaging system 101 can be substantially extended.

More specifically, the depth of field can be substantially extended from the range indicated by DP1 in FIG. 9 to the range indicated by DP2. As used herein “substantially extend” means that the depth of field is optically not extended but the depth of field is apparently extended to a range in which an image of a subject essentially located outside the range of depth of field is captured as if it was located within the range of depth of field, through image processing performed by the information processing system 100. In other words, when the subject is located at a position away from the first imaging system 101 by the object distance indicated by D1, a processing target image having a blur is output from the first imaging system 101. However, since the position is located within the range of substantial depth of field indicated by DP2, the processing target image is corrected to a corrected image in focus, and the corrected image is output from the information processing system 100. In the following description, the substantial depth of field indicated by DP2 in FIG. 9 that is extended using the trained model 120 in the present embodiment is referred to as target depth of field. The corrected image in focus here is not necessarily in focus in a strict sense. For example, even when the output corrected image is partially blurred, the user may determine that the function of the information processing system 100 suffices as long as, for example, treatment using the endoscopic scope 310 is practicable. In other words, the distance of the target depth of field in the present embodiment is a distance that is wider than the distance of depth of field optically defined but is variable according to the user's acceptance level. Therefore, DP2 in FIG. 9 is depicted for the sake of convenience and is not intended to depict a constant length. This is applicable in the following description.

The trained model 120 in the present embodiment is trained by machine learning such that a blurred image obtained by capturing an image of a subject located in the range indicated by DP10 in FIG. 9, as a difference between the target depth of field indicated by DP2 and the depth of field indicated by DP1, is corrected to an image in focus. In other words, the distance indicated by DP10 is a distance necessary for machine learning.

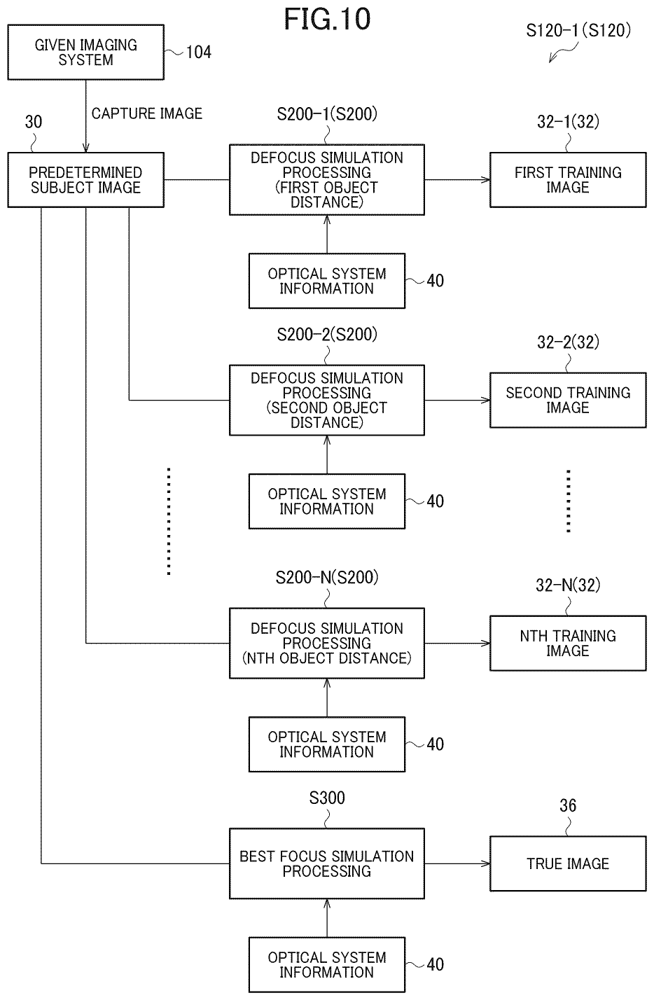

The technique in the image data generation processing (step S120) for generating the training image 32 and the true image 36 necessary for the machine learning will now be described with reference to FIG. 10. The technique in the image data generation processing is not limited to that of FIG. 10 and various modifications may be implemented as described later. The image data generation processing illustrated in FIG. 10 can also be called step S120-1.

It is assumed that the predetermined subject image 30 in the present embodiment is captured at an object distance at which an imaging system that captures the image is focused, in any of examples.

The training device processing section 16 generates the training image 32 by performing the defocus simulation processing (step S200) for the predetermined subject image 30 captured by the given imaging system 104. In the following description, the defocus simulation processing for generating, for example, the first training image 32-1 can also be called step S200-1. Similarly, the defocus simulation processing for generating the Nth training image 32-N can also be called step S200-N. This is applicable to steps S202, S204, S206, S208, S210, S220, and S230 described later. For example, in generating the first training image 32-1 through the defocus simulation processing (step S200-1), the training device processing section 16 selects information on the first object distance from the read optical system information 40. Similarly, in generating the second training image 32-2 in step S200-2, the training device processing section 16 selects information on the second object distance from the read optical system information 40. In other words, in the present embodiment, this can be generalized to the following representation: the optical system information 40 corresponding to the Nth training image 32-N is the Nth object distance, and in generating the Nth training image 32-N, the training device processing section 16 selects information on the corresponding Nth object distance from the optical system information 40. In the following description, the defocus simulation processing will be described taking the processing for generating the first training image 32-1 as an example, but the processing is similar to a case where the second training image 32-2 to the Nth training image 32-N are generated.

Further, the training device processing section 16 generates the true image 36 by performing the best focus simulation processing (step S300) for the predetermined subject image 30. For example, the training device processing section 16 selects information on an object distance at which the first imaging system 101 is focused, from the read optical system information 40. The information on the object distance at which the first imaging system 101 is focused is a distance in design from the first imaging system 101 to the point indicated by P1 in FIG. 9, for example, as indicated by D3, which is an object distance corresponding to what is called a best focus condition.

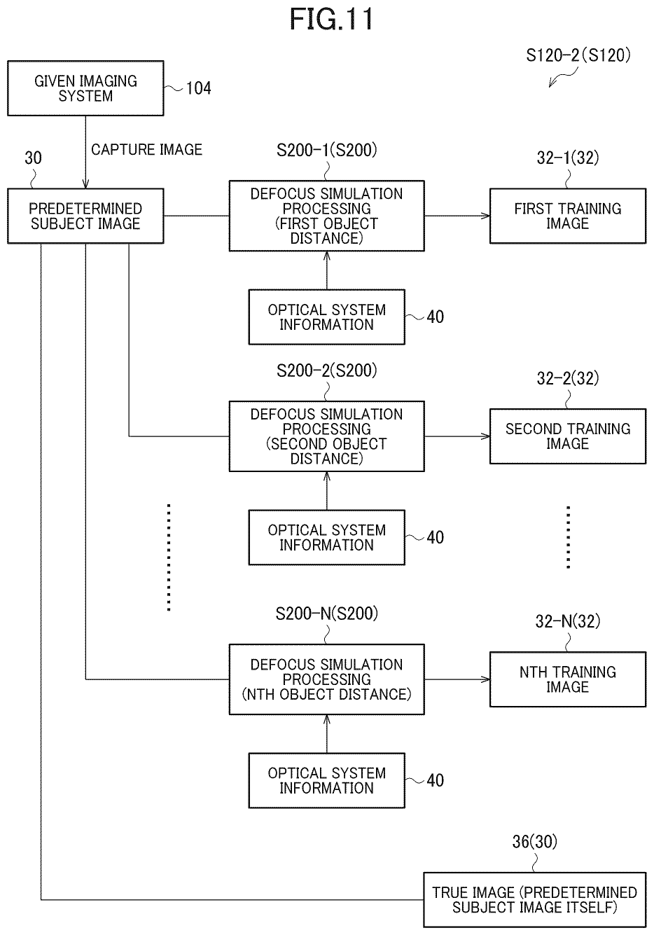

The image data generation processing in the present embodiment may be as illustrated in FIG. 11. The image data generation processing illustrated in FIG. 11 can also be called step S120-2. Processing similar to that of FIG. 10 will not be further elaborated.

Step S120-2 in FIG. 11 differs from step S120-1 in FIG. 10 in that the best focus simulation processing (step S300) is not performed, and that the true image 36 is the predetermined subject image 30 itself. This is because if the predetermined subject image 30 is an image captured at an object distance at which the given imaging system 104 is focused, the predetermined subject image 30 can be used as the true image 36.

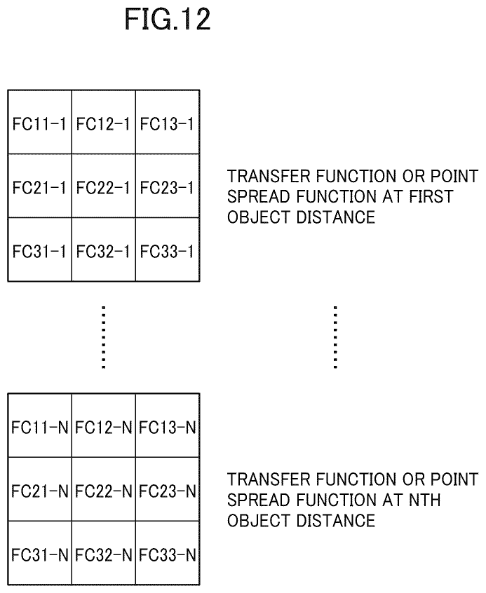

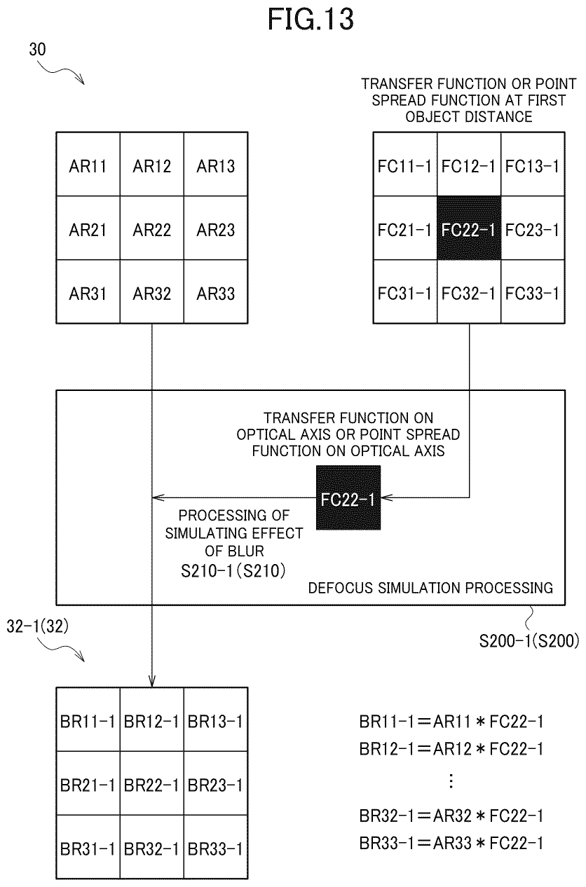

The defocus simulation processing (step S200) will be described with reference to FIG. 12 and FIG. 13. The optical system information 40 to be read in performing the defocus simulation processing (step S200) includes information on a transfer function or a point spread function. The transfer function or the point spread function changes depending on an amount of defocus in the optical axis direction and an image height in a plane perpendicular to the optical axis. For example, at the first object distance, it is assumed that a region in a direction perpendicular to the optical axis and with the same size as the predetermined subject image 30 is divided into regions FC11-1, FC12-1, FC13-1, FC21-1, FC22-1, FC23-1, FC31-1, FC32-1, and FC33-1. In this case, the transfer function or the point spread function at the first object distance may exhibit a value different for each of the divided regions. Similarly, for example, at the Nth object distance, it is assumed that a region in a direction perpendicular to the optical axis and with the same size as the predetermined subject image 30 is divided into regions FC11-N, FC12-N, FC13-N, FC21-N, FC22-N, FC23-N, FC31-N, FC32-N, and FC33-N. In this case, the transfer function or the point spread function at the Nth object distance may exhibit a value different for each of the divided regions. Further, the transfer function or the point spread function of the region FC11-1 and the transfer function or the point spread function of the region FC11-N may exhibit different values. This is applicable to cases between the regions FC12-1 and FC12-N, . . . , between the regions FC33-1 and FC33-N. In this way, if the training image group 32G is a set of N training images 32, as illustrated in FIG. 12, information on transfer function or point spread function is enormous in performing machine learning.

In this respect, in the present embodiment, the transfer function or the point spread function on the optical axis is used in performing machine learning. In the present embodiment, it is assumed that the region FC22-1 is a region where the optical axis of the first imaging system 101 passes. In other words, the transfer function or the point spread function in the region FC22-1 is the transfer function or the point spread function on the optical axis of the first imaging system 101 at the first object distance. Similarly, the transfer function or the point spread function in the region FC22-N is the transfer function or the point spread function on the optical axis of the first imaging system 101 at the Nth object distance. The transfer function or the point spread function is divided into nine parts in FIG. 12, but this is illustrated only by way of example. This is applicable to FIG. 13. For example, the regions FC22-1 to FC22-N in FIG. 12 are a set including a predetermined number of pixels in each of the vertical direction and the horizontal direction but may be one pixel. In other words, the transfer function or the point spread function on the optical axis in the present embodiment is the transfer function or the point spread function in at least one of the area of one pixel passing through the optical axis or the area of a predetermined number of pixels including the one pixel.

As illustrated in FIG. 13, in the defocus simulation processing (step S200), processing of simulating the effect of a blur (step S210) is performed for the predetermined subject image 30, based on the transfer function on the optical axis or the point spread function on the optical axis of the first imaging system 101. Step S210 will be detailed later. In other words, step S210 is also performed for a region other than the region on the optical axis of the predetermined subject image 30, based on the transfer function on the optical axis or the point spread function on the optical axis of the first imaging system 101. For example, it is assumed that the predetermined subject image 30 is divided into nine regions, namely, regions AR11, AR12, AR13, AR21, AR22, AR23, AR31, AR32, and AR33, in the same manner as in FIG. 12. For example, in a case where the first training image 32-1 is generated, the training device processing section 16 performs computation in step S210-1 for the region AR11 using the transfer function or the point spread function on the optical axis indicated in FC22-1 in FIG. 12. This computation is briefly referred to as AR11*FC22-1 in the following description and the illustration in FIG. 13. This is applicable to computation in step S210 and the like using other regions. Here “*” denotes convolution, for example, when PSF is used as the point spread function, which will be detailed later. Further, for example, when OTF is used as the transfer function, “*” means that a frequency characteristic of Fourier transform of the region AR11 is multiplied by OTF of the region FC22-1.

In addition, the training device processing section 16 also performs step S210-1 for the regions AR12 to AR33, using the transfer function or the point spread function on the optical axis indicated in FC22-1. In other words, the training device processing section 16 performs AR12*FC22-1, AR13*FC22-1, AR21*FC22-1, AR22*FC22-1, AR23*FC22-1, AR31*FC22-1, AR32*FC22-1, and AR33*FC22-1, which are partially omitted in FIG. 13. In this way, the training device processing section 16 divides the same region as the predetermined subject image 30 into a desired number of regions and performs step S210 using the transfer function or the point spread function of one of the divided regions.

Similarly, it is assumed that the generated first training image 32-1 is divided into nine regions, namely, regions BR11-1, BR12-1, BR13-1, BR21-1, BR22-1, BR23-1, BR31-1, BR32-1, and BR33-1. The region BR11-1 corresponds to the result of step S210-1 performed for the region AR11 described above. In other words, as illustrated in FIG. 13, BR11-1=AR11*FC22-1. Similarly, BR12-1=AR12*FC22-1, BR13-1=AR13*FC22-1, BR21-1=AR21*FC22-1, BR22-1=AR22*FC22-1, BR23-1=AR23*FC22-1, BR31-1=AR31*FC22-1, BR32-1=AR32*FC22-1, and BR33-1=AR33*FC22-1.

This technique is applicable to a case where the Nth training image 32-N is generated. In other words, although not illustrated in the drawings, the training device processing section 16 performs BR11-N=AR11*FC22-N, BR12-N=AR12*FC22-N, . . . , BR22-N=AR22*FC22-N, . . . , BR32-N=AR32*FC22-N, and BR33-N=AR33*FC22-N. Based on the foregoing, the defocus simulation processing (step S200) is performed for the region (BR22) on the optical axis of the first imaging system 101 and the regions other than on the optical axis (BR11, . . . , BR21, BR23, . . . . BR33) in each training image 32, based on the transfer function or the point spread function on the optical axis (FC22).

The transfer function in the present embodiment may be called optical transfer function or OTF. OTF is an abbreviation of optical transfer function. The point spread function in the present embodiment may be called point spread function or PSF. PSF is an abbreviation of point spread function.

OTF is the result of Fourier transform of PSF. In other words, PSF is the result of inverse Fourier transform of OTF. Further, OTF is a complex function, and the absolute value of OTF is referred to as modulation transfer function, amplitude transfer function, or MTF. MTF is an abbreviation of modulation transfer function.

Based on the foregoing, the information processing system 100 according to the present embodiment includes the memory section 110 that stores the trained model 120 trained by machine learning with a data set including the training image group 32G and the true image 36, and the processing section 130 that uses the trained model 120 to correct a blur caused by defocus of the first imaging system 101 in the processing target image which is an image captured by the first imaging system 101. The training image group 32G includes a plurality of training images 32 generated by performing the defocus simulation processing (step S200) that simulates, for the predetermined subject image 30 in which the given imaging system 104 is focused on a predetermined subject of which image is captured by the given imaging system 104, the effect of a blur caused by defocus of the first imaging system 101, based on the transfer function or the point spread function of the first imaging system 101 at a plurality of object distances. The defocus simulation processing is performed for the region on the optical axis of the first imaging system 101 and the regions other than on the optical axis in each training image 32 of a plurality of training images 32, based on the transfer function or the point spread function on the optical axis. The true image 36 is an image generated by performing the best focus simulation processing (step S300) that simulates, for the predetermined subject image 30, a state in which the first imaging system 101 is focused, based on the transfer function or the point spread function at an object distance at which the first imaging system 101 is focused, or the predetermined subject image 30 itself. The trained model 120 is trained by machine learning so that each training image 32 is the true image 36.

In this way, the information processing system 100 according to the present embodiment, which includes the memory section 110 that stores the trained model 120 and the processing section 130, can output a corrected image in which the effect of a blur is corrected, even when the processing target image captured by the first imaging system 101 includes the effect of a blur caused by defocus. As a result, the depth of field of the first imaging system 101 can be substantially extended. Further, since the training image group 32G and the true image 36 are created in advance based on the predetermined subject image 30 captured by the given imaging system 104, the trained model 120 trained by machine learning in advance can be used when a subject associated with the processing target image is a subject of which image is captured by the first imaging system 101 for the first time. Further, the defocus simulation processing (step S200) is performed for the region on the optical axis of the first imaging system 101 and the regions other than on the optical axis in each training image 32, based on the transfer function or the point spread function on the optical axis, thereby reducing the volume of information necessary for the defocus simulation processing (step S200). As a result, the trained model 120 can be created with an appropriate scale of the neural network NN necessary for machine learning. This facilitates implementation of the trained model 120 in the information processing system 100.

The technique of the present embodiment can also be implemented as the trained model 120. In other words, the trained model 120 in the present embodiment is used by the information processing system 100 including the memory section 110 that stores the trained model 120, the input section 140, the processing section 130, and the output section 150, and is trained by machine learning using a data set including the training image group 32G and the true image 36. The training image group 32G includes a plurality of training images 32 generated by performing the defocus simulation processing that simulates the effect of a blur caused by defocus of the first imaging system 101, for the predetermined subject image 30 in which the given imaging system 104 is focused on a predetermined subject of which image is captured by the given imaging system 104, based on the transfer function or the point spread function of the first imaging system 101 at a plurality of object distances. The defocus simulation processing is performed for the region on the optical axis of the first imaging system 101 and the regions other than on the optical axis in each training image 32 of a plurality of training images 32, based on the transfer function or the point spread function on the optical axis. The true image 36 is an image generated by performing the best focus simulation processing that simulates, for the predetermined subject image 30, a state in which the first imaging system 101 is focused, based on the transfer function or the point spread function at an object distance at which the first imaging system 101 is focused, or the predetermined subject image 30 itself. The trained model 120 is trained by machine learning so that each training image 32 is the true image 36. The input section 140 inputs the processing target image, which is an image captured by the first imaging system 101, to the trained model 120. The processing section 130 performs the correction processing of correcting a blur caused by defocus of the first imaging system 101 in the processing target image, using the trained model 120. The output section 150 outputs a corrected image produced by the correction processing. In this way, effects similar to those described above can be achieved.

The technique of the present embodiment can also be implemented as an information processing method. In other words, the information processing method according to the present embodiment corrects a blur caused by defocus of the first imaging system 101 in the processing target image which is an image captured by the first imaging system 101, using the trained model 120 trained by machine learning with a data set including the training image group 32G and the true image 36. The training image group 32G includes a plurality of training images 32 generated by performing the defocus simulation processing that simulates the effect of a blur caused by defocus of the first imaging system 101, for the predetermined subject image 30 in which the given imaging system 104 is focused on a predetermined subject of which image is captured by the given imaging system 104, based on the transfer function or the point spread function of the first imaging system 101 at a plurality of object distances. The defocus simulation processing is performed for the region on the optical axis of the first imaging system 101 and the regions other than on the optical axis in each training image 32 of a plurality of training images 32, based on the transfer function or the point spread function on the optical axis. The true image 36 is an image generated by performing the best focus simulation processing that simulates, for the predetermined subject image 30, a state in which the first imaging system 101 is focused, based on the transfer function or the point spread function at an object distance at which the first imaging system 101 is focused, or the predetermined subject image 30 itself. The trained model 120 is trained by machine learning so that each training image 32 is the true image 36. In this way, effects similar to those described above can be achieved.

The technique of the present embodiment can also be implemented as an information storage medium that stores the trained model 120. In this way, the training model 20 trained by machine learning by the training device 10 can be stored in the information storage medium. With this configuration, the information storage medium is connected to the information processing system 100, whereby the training model 20 can be updated as the latest trained model 120. As a result, effects similar to those described above can be achieved under a predetermined situation. The predetermined situation includes, for example, a situation in which the location of the training device 10 is distant from the location of the information processing system 100, a situation in which data communication fails between the training device 10 and the information processing system 100, and the like.

The technique of the present embodiment can also be implemented as the endoscope system 300. For example, the endoscope system 300 according to the present embodiment includes a processor unit 200 including the information processing system 100 described above and an endoscopic scope 310 connected to the processor unit 200 to capture a processing target image. In this way, the endoscope system 300 including the information processing system 100 having the aforementioned effects can be constructed.

More specifically, the endoscope system 300 may have, for example, a configuration example as illustrated in FIG. 14. The endoscope system 300 includes the endoscopic scope 310, an operation section 320, a display section 330, and the processor unit 200. The processor unit 200 includes a storage section 210, a control section 220, and the information processing system 100. The information processing system 100 in FIG. 14 further includes a storage interface 160 in addition to the configuration described above with reference to FIG. 2. A configuration similar to that of FIG. 2 will not be further elaborated.

The endoscopic scope 310 includes an imaging device at a not-illustrated distal end thereof. The imaging device includes the first imaging system 101. The distal end of the endoscopic scope 310 is inserted into a body cavity. The imaging device captures an image of an abdominal cavity, and captured image data is transmitted from the endoscopic scope 310 to the processor unit 200. The operation section 320 is a device for the user to operate the endoscope system 300 and includes, for example, a button or a dial, a foot switch, or a touch panel. The display section 330 is a device that displays an image captured by the endoscopic scope 310. The display section 330 is, for example, a liquid crystal display but may be hardware integrated with the operation section 320, such as a touch panel.

The processor unit 200 performs control in the endoscope system 300 and processing such as image processing. For example, the control section 220 performs switching of a mode of the endoscope system 300, a zoom operation, switching of display, or the like, based on information input from the operation section 320, whereby the function as the processor unit 200 is implemented. The storage section 210 records an image captured by the endoscopic scope 310. The storage section 210 is, for example, a semiconductor memory, a hard disk drive, an optical disk drive, or the like.

In the configuration example illustrated in FIG. 14, a connector connected to a cable of the endoscopic scope 310 or an interface circuit or the like that receives imaging data is incorporated in the input section 140 to implement a function of receiving imaging data from the endoscopic scope 310. However, the processor unit 200 may further include an interface circuit for receiving imaging data.

The storage interface 160 is an interface for accessing the storage section 210. The storage interface 160 records image data received by the input section 140 into the storage section 210. When replaying the recorded image data, the storage interface 160 reads the image data from the storage section 210 and transmits the image data to the processing section 130. The processing section 130 performs the processing described above with reference to FIG. 3 on the image data from the input section 140 or the storage interface 160 as the processing target image. With this configuration, the processing section 130 outputs the corrected image via the output section 150, and the corrected image in focus appears on the display section 330.

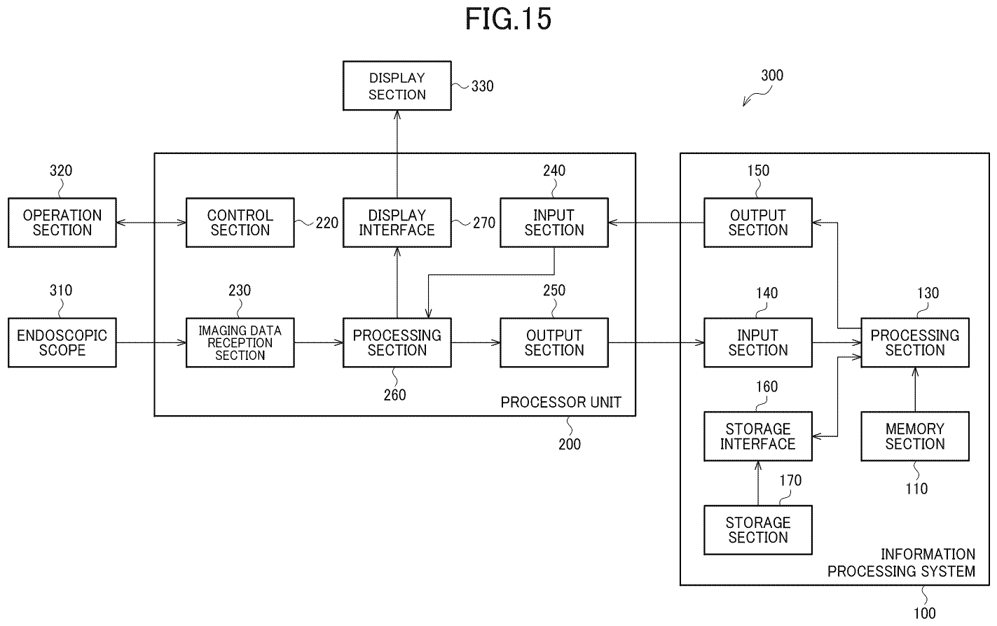

The endoscope system 300 according to the present embodiment may have, for example, a configuration example illustrated in FIG. 15. The configuration example in FIG. 15 differs from the configuration example in FIG. 14 in that the information processing system 100 and the processor unit 200 are provided separately. The information processing system 100 and the processor unit 200 may be connected by device-to-device communication such as a universal serial bus (USB), or by network communication such as a local area network (LAN) and a wide area network (WAN). The information processing system 100 includes one or more information processing devices. In a case where the information processing system 100 includes a plurality of information processing devices, the information processing system 100 may be a cloud system in which a plurality of PCs, a plurality of servers, and the like connected via a network perform parallel processing. A storage section 170 in FIG. 15 corresponds to the storage section 210 in FIG. 14.

The processor unit 200 includes a control section 220, an imaging data reception section 230, an input section 240, an output section 250, a processing section 260, and a display interface 270. The imaging data reception section 230 is configured with an interface circuit or the like similar to the input section 140 in FIG. 14 and receives imaging data from the endoscopic scope 310. The processing section 260 transmits image data received by the imaging data reception section 230 to the information processing system 100 via the output section 250. The information processing system 100 performs the processing in FIG. 3 on the received image data as the processing target image and generates a corrected image. The input section 240 receives the corrected image transmitted from the information processing system 100 via the output section 150, and outputs the corrected image to the processing section 260. The processing section 260 outputs the corrected image to the display section 330 via the display interface 270. As a result, the corrected image appears on the display section 330. The display interface 270 in FIG. 15 is configured with hardware similar to the output section 150 in FIG. 14 and implements a function similar to the output section 150 in FIG. 14. In FIG. 15, the input section 140 and the output section 150 of the information processing system 100 may be configured with separate interfaces, but the functions of the input section 140 and the output section 150 may be implemented by a single input/output interface. This is applicable to the input section 240 and the output section 250 of the processor unit 200.

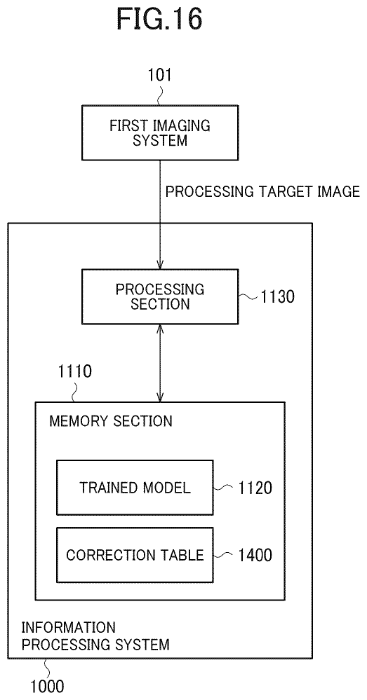

The technique of the present embodiment is not limited to the foregoing and various modifications may be implemented. For example, the endoscope system 300 according to the present embodiment may include the endoscopic scope 310 described above with reference to FIG. 14 and the like and an information processing system 1000 illustrated in FIG. 16. The information processing system 1000 illustrated in FIG. 16 includes a memory section 1110 and a processing section 1130. The memory section 1110 includes a trained model 1120 and a correction table 1400. The memory section 1110 in FIG. 16 can be implemented by a memory similar to the memory section 110 in FIG. 1, and the processing section 1130 can be implemented by a processor similar to the processing section 130 in FIG. 1. The trained model 1120 and the correction table 1400 will be detailed later.

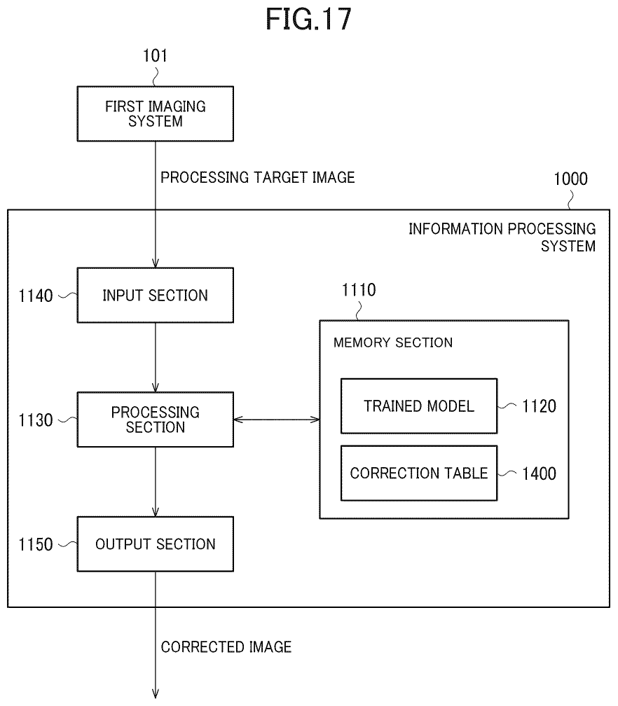

FIG. 17 is a block diagram illustrating a configuration example of the information processing system 1000 in more detail. In FIG. 17, the information processing system 1000 further includes an input section 1140 and an output section 1150, in addition to the memory section 1100 and the processing section 1130 described above. The input section 1140 in FIG. 17, similar to the input section 140 in FIG. 2, is an image data interface that receives image data as a processing target image from the first imaging system 101. The output section 1150 in FIG. 17, similar to the output section 1150 in FIG. 2, is an interface that transmits a corrected image to the outside and serves the function of the output section 1150 such that output data from the trained model 1120 is the corrected image transmitted by the output section 1150.

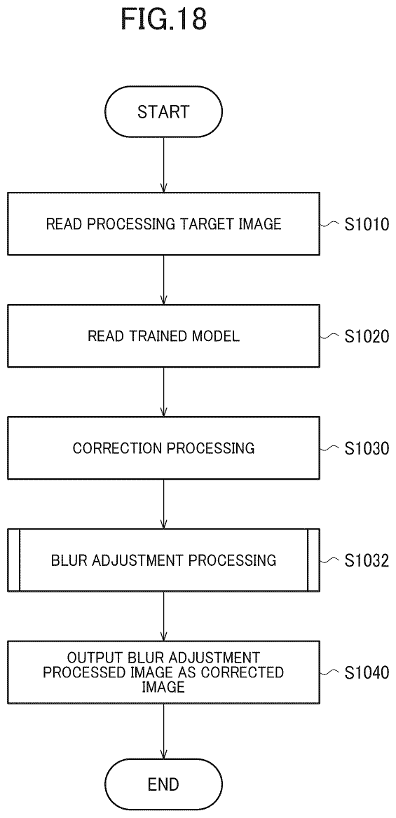

The processing example related to the technique of the present embodiment may be performed according to a modified flowchart illustrated in FIG. 18 instead of the flowchart illustrated in FIG. 3. In FIG. 18, the processing section 1130 reads the processing target image (step S1010) and reads the trained model (step S1020). Step S1010 in FIG. 18 is processing corresponding to step S10 in FIG. 3, and step S1020 in FIG. 18 is processing corresponding to step S20 in FIG. 3. In other words, specifically, for example, the processing section 1130 performs processing of inputting the processing target image received via the input section 1140 to the trained model 1120 read from the memory section 1110.

The processing section 1130 thereafter performs correction processing (step S1030) and blur adjustment processing (step S1032), and outputs a blur adjustment processed image 1034 as a corrected image (step S1040). In this way, the corrected image in FIG. 17 differs from the corrected image in FIG. 2 in that it is the blur adjustment processed image 1034 generated by further performing blur adjustment processing (step S1032) for the output image 134 generated by performing correction processing (step S1030) for the processing target image.

The machine learning for generating the trained model 1120 in FIG. 16 will now be described. The trained model 1120 in FIG. 16 is trained by machine learning with a data set including an object distance label in addition to the training image 32 and the true image 36 described above.

More specifically, the object distance label in the present embodiment refers to an estimated object distance label 1070 and a true object distance label 1076 described later.

For example, the training model 1020 described later can be updated as the trained model 1120 by performing machine learning using the training device 10 or the like described above with reference to FIG. 4. At least a part of the training model 1020 includes the neural network NN described above, which is similar to the training model 20 in FIG. 4.

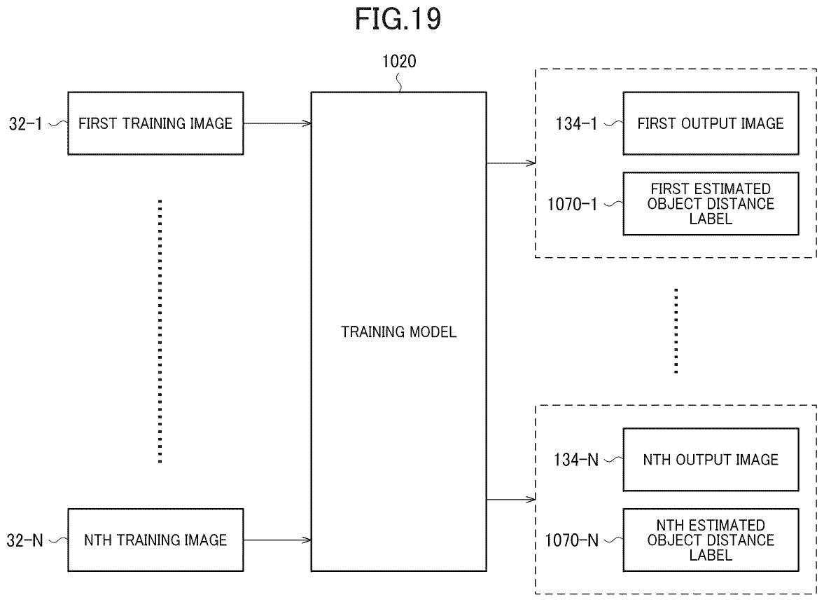

As illustrated in FIG. 19, the training image 32 described above with reference to FIG. 5 and the like is input as input data to the training model 1020. The types of training image 32 are as many as the number of object distances. In other words, as described above with reference to FIG. 10, the first training image 32-1 is generated by performing defocus simulation processing (step S200-1) on the transfer function or the point spread function corresponding to the first object distance for the predetermined subject image 30. Similarly, the Nth training image 32-N is generated by performing defocus simulation processing (step S200-N) on the transfer function or the point spread function corresponding to the Nth object distance for the predetermined subject image 30. In this way, it can be generalized that in the learning phase, machine learning is performed by inputting the Nth training image 32-N as input data to the training model 1020.

In FIG. 13, the transfer function or the point spread function on the optical axis is used for the defocus simulation processing (step S200). This is applicable to the defocus simulation processing (step S200) to be used to generate the training image 32 to be input to the training model 1020 in FIG. 19. As a result, the network scale necessary for generating the trained model 1120 in FIG. 16 and the like can be reduced.

The output image 134 is then output from the training model 1020 through computation by the neural network NN. The output image 134 is estimation result data derived by the training model 1020 from the training image 32, which is estimation target data, and is similar to the output image 34 described above with reference to FIG. 5.

When the training image 32 is input to the training model 1020, the object distance corresponding to the transfer function or the point spread function used for the input training image 32 is estimated through computation by the neural network NN. As a result, the estimated object distance label 1070 associated with the estimated object distance is output together with the output image 134 described above. In other words, the estimated object distance label 1070 is a class classification result. Based on the foregoing, in the learning phase, as a result of inputting the Nth training image 32-N as input data to the training model 1020, the Nth output image 134-N and the Nth estimated object distance label 1070-N are output as output data from the training model 1020.

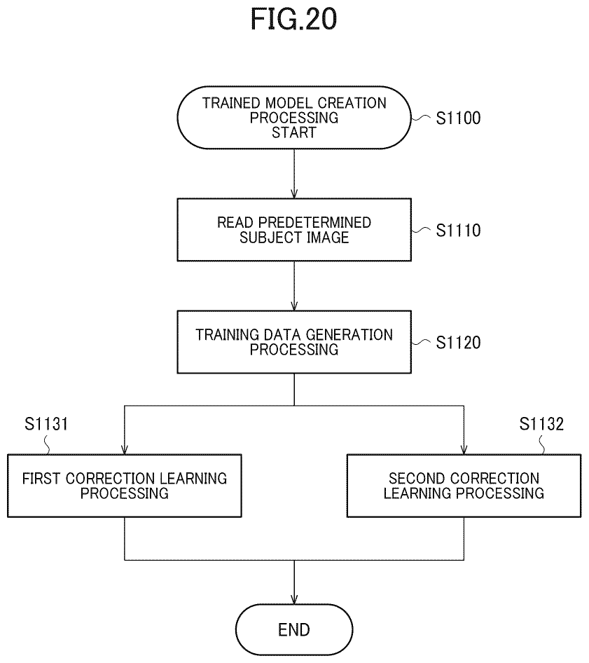

FIG. 20 is a flowchart illustrating a processing example of trained model creation processing (step S1100) associated with the trained model 1120. After reading a predetermined subject image (step S1110), the training device processing section 16 performs training data generation processing (step S1120). For example, the training device processing section 16 reads the predetermined subject image 30 from the training device memory section 18 and generates the training image 32 and the true image 36 using the predetermined subject image 30, as described above with reference to FIG. 7. The training device processing section 16 also generates the true object distance label 1076 based on the object distance corresponding to the transfer function or the point spread function associated with the defocus simulation processing (step S200) used to generate the training image 32.

In FIG. 10 and FIG. 11, the true image 36 is an image generated by performing the best focus simulation processing (step S300) for the predetermined subject image 30 or the predetermined subject image 30 itself. This is applicable to the true image 36 generated by step S1120 in FIG. 20.

The training device processing section 16 then performs first correction learning processing (step S1131) and second correction learning processing (step S1132) and terminates the flow. For example, the training device processing section 16 performs processing of reading the training model 1020 from the training device memory section 18 and inputting to the training model 1020 the input data with the training image 32 generated in the training data generation processing (step S1120). As a result, the output image 134 and the estimated object distance label 1070 are output as output data from the training model 1020 as described above with reference to FIG. 19. The training device processing section 16 then performs machine learning based on the output image 134 and the true image 36 as the first correction learning processing (step S1131). The first correction learning processing (step S1131) is similar to the correction learning processing (step S130) described above with reference to FIG. 7 and FIG. 8, and detailed description and illustration are omitted.

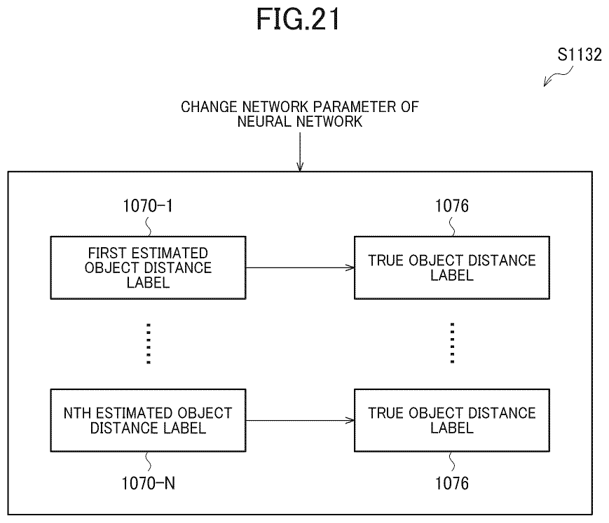

The training device processing section 16 also performs machine learning processing as the second correction learning processing (step S1132), based on the estimated object distance label 1070 and the true object distance label 1076 generated in step S1120. For example, the training device processing section 16 changes network parameters of the neural network NN so that the first estimated object distance label 1070-1 to the Nth estimated object distance label 1070-N are the true object distance labels 1076, as illustrated in FIG. 21. The meaning of changing the network parameters of the neural network NN here are described above with reference to FIG. 7.

More specifically, for example, it is assumed that the training image 32 obtained by performing the defocus simulation processing (step S200) for the predetermined subject image 30 using the transfer function or the point spread function at the Mth object distance is input to the training model 1020. In this case, the true object distance label 1076 corresponding to the Mth object distance is generated by step S1120.

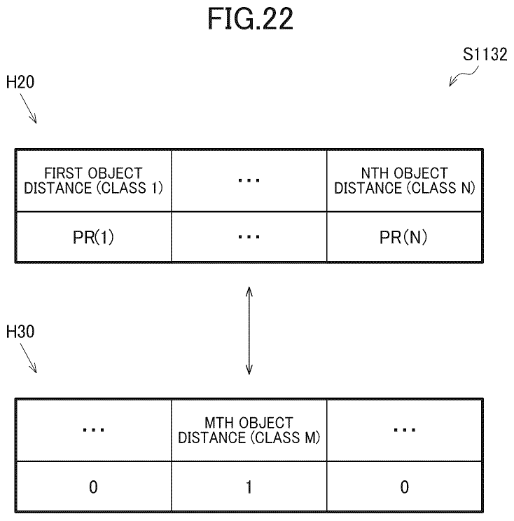

The estimated object distance label 1070 output from the training model 1020 is then classified into a class according to the estimated object distance. For example, if the object distance associated with the estimated object distance label 1070 output from the training model 1020 is the first object distance, it is classified as class 1. Similarly, if the object distance associated with the estimated object distance label 1070 output from the training model 1020 is the Nth object distance, it is classified as class N. The same processing is repeated to construct, for example, a class classification table indicated by H20 in FIG. 22. In the table indicated by H20, a probability PR(1) is the probability that the estimated object distance associated with the estimated object distance label 1070 is the first object distance. Similarly, a probability PR(N) is the probability that the estimated object distance associated with the estimated object distance label 1070 is the Nth object distance.

Then, in the second correction learning processing (step S1132), the processing of calculating an error function based on the table indicated by H20 and a table indicated by H30 and the processing of updating a weighting factor of the neural network NN so that the error function is reduced are performed. The table indicated by H30 is a table in which the probability that the estimated object distance associated with the estimated object distance label 1070 is the Mth object distance associated with the true object distance label 1076 is 1, and the probability that the estimated object distance associated with the estimated object distance label 1070 is an object distance other than the Mth object distance is 0.

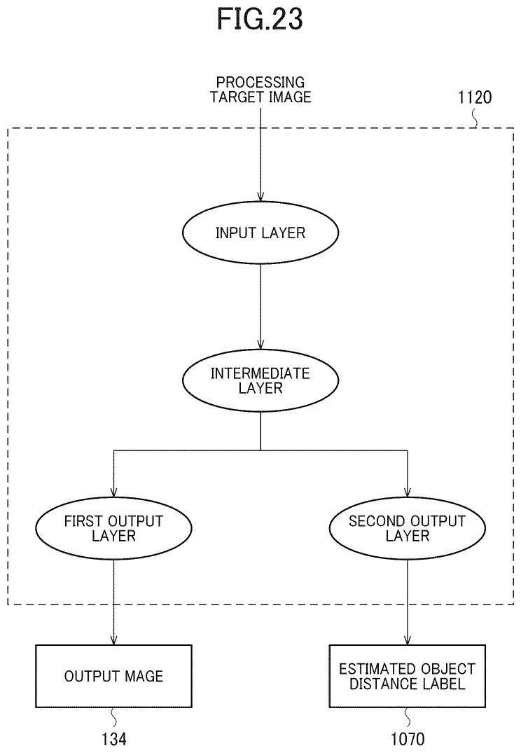

Through the optimization of the parameters of the neural network NN in this manner, the training model 1020 is updated as the trained model 1120. Inference using the updated trained model 1120 is then performed as illustrated in FIG. 23. In other words, when a processing target image is input to the input layer of the neural network NN included in the trained model 1120, computation is performed by the intermediate layer. Based on the computation result by the intermediate layer, the output image 134 is output from a first output layer and the estimated object distance label 1070 is output from a second output layer. In other words, the trained model 1120 in FIG. 23 is constructed with one neural network NN having an output layer split into the first output layer and the second output layer, and the output image 134 and the estimated object distance label 1070 are output from the one neural network NN.

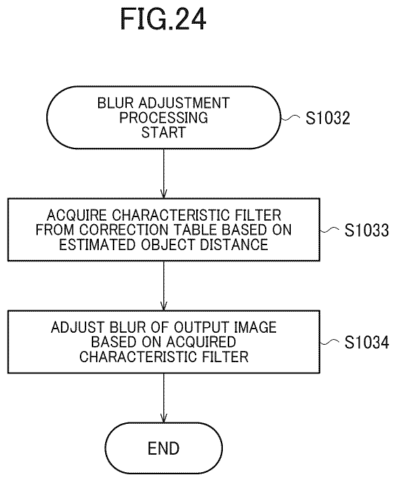

The blur adjustment processing (step S1032) in FIG. 18 will be described in more detail using the flowchart in FIG. 24. The processing section 1130 performs processing of acquiring a characteristic filter from the correction table 1400 based on the estimated object distance (step S1033) and performs processing of adjusting a blur of the output image 134 based on the acquired characteristic filter (step S1034).

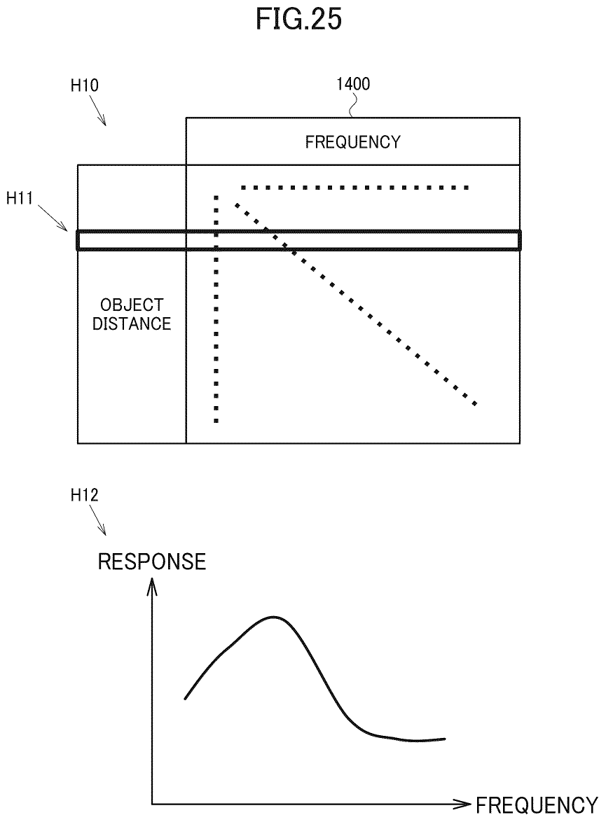

The correction table 1400 is a table in which object distances are associated with filter characteristics for blur correction. More specifically, for example, as indicated by H10 in FIG. 25, the table is a set of correction factors according to combinations of the object distance associated with the estimated object distance label 1070 output from the trained model 1120 and the frequency at image height perpendicular to the optical axis. The “object distance” in a row of the correction table 1400 is a simplified representation of the object distance associated with the estimated object distance label 1070 output from the trained model 1120.

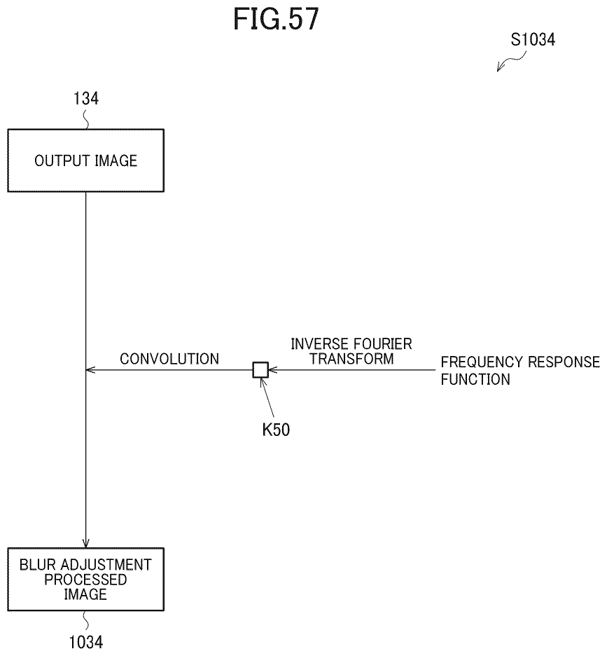

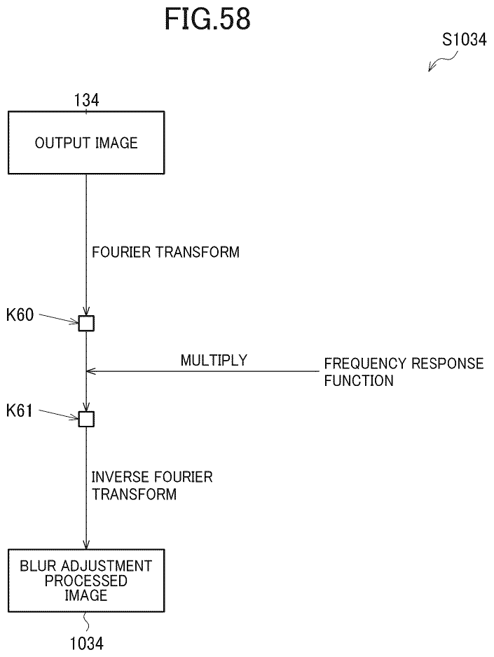

For example, in step S1033, the processing section 1130 searches the correction table 1400 for correction factor data associated with the same object distance as the object distance of the estimated object distance label 1070 generated in the correction processing (step S1030). Then, in step S1034, the processing section 1130 adjusts a blur for the output image 134 using the retrieved correction factor. For example, if the processing section 1130 retrieves correction factor data in a row indicated by H11, conversion into a frequency response function indicated by H12 may be performed based on the retrieved correction factor data. In this case, the frequency response function indicated by H12 corresponds to the characteristic filter in step S1033. Then, in step S1034, the processing section 1130 performs predetermined filter processing for the output image 134 using the frequency response function indicated by H12. The characteristic filter associated with step S1033 is not limited to the frequency response function, and details will be described later with reference to FIG. 57 and FIG. 58.

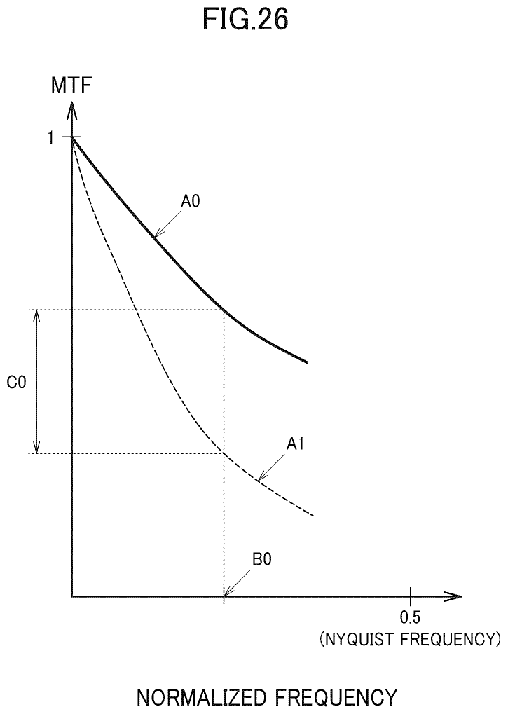

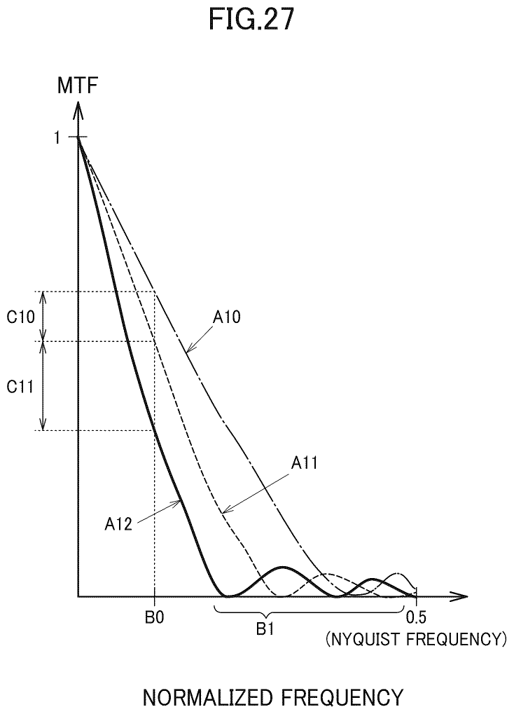

The frequency on the horizontal axis of the graph indicated by H12 in FIG. 25 is a simplified representation of a normalized frequency. In other words, the horizontal axis of the graph indicated by H12 in FIG. 25 is the same as the horizontal axes of the graphs in FIG. 26 and FIG. 27 described later.

In this way, the blur adjustment processed image 1034, which is an image obtained by the processing section 1130 performing blur adjustment processing (step S1032) for the output image 134, is output as a corrected image from the output section 1150 in step S1040 in FIG. 18. The blur adjustment processed image 1034 is output to a predetermined display device. However, the output destination is not limited to this and the corrected image may be output to a storage device of an external device or the like. Specifically, the predetermined display device is, for example, the display section 330 illustrated in FIG. 14 and the like. This is applicable to the information processing system 100 described above with reference to FIG. 2.

Based on the foregoing, the information processing system 1000 according to the present embodiment includes the memory section 1110 and the processing section 1130. The memory section 1110 stores the trained model 1120 trained by machine learning with a data set including the training image 32, the true image 36, and the object distance label, and the correction table 1400 in which an object distance which is a distance between an imaging system and a subject is associated with a filter characteristic for blur correction. The training image 32 is generated by performing the defocus simulation processing that simulates the effect of a blur caused by defocus of the first imaging system 101, for the predetermined subject image 30 in focus captured by the given imaging system 104, based on the transfer function or the point spread function of the first imaging system 101 at a predetermined object distance. The defocus simulation processing is performed for the region on the optical axis and the regions other than on the optical axis of the first imaging system 101 in the training image, based on the transfer function or the point spread function on the optical axis. The true image 36 is an image generated by performing the best focus simulation processing that simulates, for the predetermined subject image 30, a state in which the first imaging system 101 is focused, based on the transfer function or the point spread function at an object distance at which the first imaging system 101 is focused, or the predetermined subject image 30 itself. The trained model 1120 is trained by machine learning so that the training image 32 is the true image 36, and trained by machine learning by applying, as the object distance label, the object distance of the transfer function or the point spread function of the first imaging system 101 used in the defocus simulation processing. The processing section 1130 uses the trained model 1120 to generate the output image 134 in which a blur of the processing target image which is an image captured by the first imaging system 101 is corrected, and estimates an object distance of the processing target image. The processing section 1130 acquires a filter characteristic associated with the estimated object distance from the correction table 1400, and performs blur adjustment processing for the output image 134 using the acquired filter characteristic.

In this way, the information processing system 1000 according to the present embodiment, which includes the memory section 110 that stores the trained model 1120 and the correction table 1400 and the processing section 130, can output a corrected image in which the effect of a blur is corrected more appropriately from the processing target image. As a result, the depth of field of the first imaging system 101 can be substantially extended. Further, the defocus simulation processing (step S200) is performed for the region on the optical axis of the first imaging system 101 and the regions other than on the optical axis in each training image 32, based on the transfer function or the point spread function on the optical axis, thereby reducing the volume of information necessary for the defocus simulation processing (step S200). As a result, the trained model 1120 can be created with an appropriate scale of the neural network NN necessary for machine learning. This facilitates implementation of the trained model 1120 in the information processing system 1000.

The technique described above can be implemented as the endoscope system 300 described above with reference to FIG. 14 or FIG. 15. In other words, the endoscope system 300 according to the present embodiment includes the information processing system 1000 described above and an endoscopic scope 310 that captures a processing target image. In this way, effects similar to those described above can be achieved.

The technique of the present embodiment may also be implemented as an image processing method. In other words, the technique of the present embodiment relates to an image processing method using the trained model 1120 trained by machine learning with a data set including the training image 32, the true image 36, and the object distance label, and the correction table 1400 in which an object distance which is a distance between an imaging system and a subject is associated with a filter characteristic for blur correction. The image processing method includes a step of using the trained model 1120 to generate the output image 134 in which a blur of the processing target image which is an image captured by the first imaging system 101 is corrected, and a step of estimating an object distance of the processing target image (step S1030). The image processing method further includes a step of acquiring a filter characteristic associated with the estimated object distance from the correction table 1400, and performing blur adjustment processing for the output image 134 using the acquired filter characteristic (step S1032). The training image 32 is generated by performing the defocus simulation processing that simulates the effect of a blur caused by defocus of the first imaging system 101, for the predetermined subject image 30 in focus captured by the given imaging system 104, based on the transfer function or the point spread function of the first imaging system 101 at a predetermined object distance. The defocus simulation processing is performed for the region on the optical axis of the first imaging system 101 and the regions other than on the optical axis in the training image 32, based on the transfer function or the point spread function on the optical axis. The true image 36 is an image generated by performing the best focus simulation processing that simulates, for the predetermined subject image 30, a state in which the first imaging system 101 is focused, based on the transfer function or the point spread function at an object distance at which the first imaging system 101 is focused, or the predetermined subject image 30 itself. The trained model 1120 is trained by machine learning so that the training image 32 is the true image 36, and trained by machine learning by applying, as the object distance label, the object distance of the transfer function or the point spread function of the first imaging system 101 used in the defocus simulation processing. In this way, effects similar to those described above can be achieved.

The technique described above may also be implemented as a program. In other words, the program according to the present embodiment causes a computer to execute the image processing method described above. The technique of the present embodiment may be implemented as a non-transitory information storage medium including the program described above. In this way, effects similar to those described above can be achieved.

In the information processing system 1000 according to the present embodiment, the trained model 1120 may be configured with one neural network NN, and the neural network NN may include an input layer to which a processing target image is input, an intermediate layer that performs computation for an output from the input layer, a first output layer that generates the output image 134 from an output from the intermediate layer, and a second output layer that estimates an object distance from an output from the intermediate layer. In this way, increase of the network scale can be prevented.

The technique of the present embodiment is not limited to the foregoing and various modifications may be implemented. For example, each object distance included in the optical system information 40 may be determined based on a difference in the corresponding MTF. For example, it is assumed that the training image group 32G includes the first training image 32-1 that undergoes step S200-1 based on the transfer function or the point spread function at the first object distance, and the second training image 32-2 that undergoes step S200-2 based on the transfer function or the point spread function at the second object distance. Further, it is assumed that the first object distance is an object distance with a larger amount of defocus, compared with the second object distance. In this case, when a spatial frequency dependence of MTF is qualitatively illustrated, the MTF based on the second object distance is as indicated by A0 in FIG. 26, and the MTF based on the first object distance is as indicated by A1. Then, for example, when a predetermined spatial frequency indicated by B0 is determined, the difference of MTF is determined as indicated by C0. The first object distance and the second object distance are then determined so that the difference of MTF indicated by C0 is smaller than a predetermined value.

The difference of MTF here is a difference of MTF between adjacent object distances. For example, it is assumed that the training image group 32G includes the first training image 32-1, the second training image 32-2, and the third training image 32-3. Further, it is assumed that the amount of defocus is larger in the order of the first object distance, the second object distance, and the third object distance. In this case, A10 in FIG. 27 indicates the frequency characteristic of the MTF at the third object distance, A11 indicates the frequency characteristic of the MTF at the second object distance, and A12 indicates the frequency characteristic of the MTF at the first object distance. Then, it is assumed that at a predetermined frequency indicated by B0, both of the difference between the MTF of A10 and the MTF of A11 as indicated by C10 and the difference between the MTF of A11 and the MTF of A12 as indicated by C11 are lower than a predetermined value. In other words, at the predetermined frequency indicated by B0, the difference between the MTF of A10 and the MTF of A12 is not considered as the predetermined value. Based on the foregoing, in the information processing system 100 according to the present embodiment, the object distance is set so that the difference in value of MTF between adjacent object distances is equal to or less than a predetermined value, at a predetermined spatial frequency of the MTF of the first imaging system 101. In this way, an appropriate combination of data sets in machine learning can be set. As described above, the trained model 120 trained by machine learning performs correction processing (step S30) so that both of the first training image 32-1 and the second training image 32-2 can be corrected to the true image 36. In addition, in order to correct the processing target image captured at an object distance between the first object distance and the second object distance to the true image 36 through the correction processing (step S30), it is preferable that the difference between the effects of a blur added to the first training image 32-1 and the second training image 32-2 is within a certain range. In this respect, the technique of the present embodiment can be applied to generate an appropriate training image group 32G, because the object distance of each training image is defined based on the MTF that indicates the effect of a blur simulated for the predetermined subject image 30. As a result, an appropriate data set can be set in machine learning.

Further, the optical system information 40 may include an object distance in the best focus condition of the first imaging system 101. The object distance in the best focus condition is specifically, for example, the distance indicated by D3 in FIG. 9. For example, the training device processing section 16 may generate the true image 36 by performing the best focus simulation processing (step S300) for the predetermined subject image 30 using the transfer function or the point spread function using the object distance in the best focus condition. In other words, in the information processing system 100 according to the present embodiment, the object distance that achieves focus is the object distance in the best focus condition. In this way, an appropriate true image 36 can be generated.

In the present embodiment, it is assumed that the transfer function or the point spread function based on the object distance has one-to-one correspondence with the training image 32. More specifically, for example, in the defocus simulation processing (step S200), it is assumed that processing of generating the third training image 32-3 is not performed using both of the transfer function or the point spread function with the first object distance and the transfer function or the point spread function with the second object distance for one predetermined subject image 30. In other words, in the information processing system 100 according to the present embodiment, each training image 32 is an image generated by performing the defocus simulation processing (step S200) for the predetermined subject image 30 based on the transfer function or the point spread function at any one object distance of a plurality of object distances. In this way, the relationship between the training images 32 in the training image group 32G can be clarified.

In a common optical system, as the spatial frequency increases, the MTF decreases and changes with periodicity. Since the MTF is an absolute value, the MTF is displayed in a folding manner in a high spatial frequency region indicated by B1 in FIG. 27. Thus, in the high spatial frequency region, it is impossible to uniquely determine which object distance one MTF corresponds to. For example, the MTF at an object distance shorter than the object distance at the near point of the target extended depth of field as indicated by P2 in FIG. 9 may be zero in the spatial frequency indicated by B0. For example, supposing that A12 in FIG. 27 is the MTF at the object distance at the near point of the target extended depth of field, a spatial frequency lower than the lowest spatial frequency at which folding occurs may be the spatial frequency indicated by B0. This is because the transfer function or the point spread function at an object distance outside the target depth of field is not used in the machine learning in the present embodiment in the first place. The target extended depth of field here does not exhibit a constant value, as described above. Based on the foregoing, in the information processing system 100 according to the present embodiment, the processing section 130 uses the trained model 120 to correct a blur caused by defocus of the first imaging system 101 for the processing target image, thereby estimating an image in which the depth of field of the first imaging system 101 is extended to the target extended depth of field wider than the depth of field. Further, the predetermined spatial frequency is a spatial frequency lower than the lowest spatial frequency at which the value of MTF at the near point of the target extended depth of field is zero. In this way, the range of the predetermined spatial frequency necessary for associating the spatial frequency with MTF in a one-to-one correspondence can be determined as appropriate.

More specifically, it is desired that the predetermined spatial frequency indicated by B0 is, for example, 0.1 as a normalized frequency. In other words, in the information processing system 100 according to the present embodiment, the predetermined spatial frequency is a spatial frequency that is ⅕ of the Nyquist frequency of an image sensor of the first imaging system 101. In this way, the spatial frequency can be associated with the MTF in a one-to-one correspondence for many optical systems. As a result, the technique of the present embodiment can be applied to the processing target image captured by various kinds of optical systems.

Further, the optical system information 40 in the present embodiment may be a combination of an object distance inside the depth of field and an object distance outside the depth of field. Specifically, for example, the optical system information 40 may include the first object distance outside the depth of field indicated by D1 in FIG. 9 and the second object distance indicated by D2. In other words, in the information processing system 100 according to the present embodiment, the first object distance of a plurality of object distances is an object distance outside the depth of field, and the second object distance of a plurality of object distances is an object distance inside the depth of field. This configuration results in data sets in which the first training image 32-1 that simulates the effect of a blur to a large degree by the defocus simulation processing (step S200) and the second training image 32-2 that simulates the effect of a blur to a small degree are combined with the true image 36. As a result, the trained model 120 trained by machine learning with these data sets can correct the processing target image having the effect of a blur in a wide range, through the correction processing (step S30).

Further, the predetermined value may be determined based on the number of training images 32 that constitute the training image group 32G. For example, in FIG. 26, it is assumed that the MTF indicated by A0 is the MTF at the object distance corresponding to the best focus condition, and the MTF indicated by A1 is the MTF at the object distance corresponding to the near point of the target depth of field. In this case, for example, when the spatial frequency is determined as the spatial frequency indicated by B0, the range of MTF is uniquely determined such that the range indicated by C0 is the largest. Then, a value obtained by dividing the range indicated by C0 based on a desired number of training images 32 is determined as the predetermined value. Based on the foregoing, in the information processing system 100 according to the present embodiment, the predetermined value is determined based on the number of object distances that can be set to two or more. In this way, the number of data sets necessary for machine learning can be determined in consideration of the load of machine learning.

As described above, since the range of MTF is uniquely determined by fixing the spatial frequency, the predetermined value may be determined in advance and the number of training images 32 may be determined based on the predetermined value. The user can determine a policy of machine learning depending on a situation.

It is desirable that the predetermined value is equal to or less than 0.2. In other words, in the information processing system 100 according to the present embodiment, the predetermined value is set to be equal to or less than 0.2. In a common optical system, when the aforementioned spatial frequency is determined in a desirable range, a possible range of MTF is presumably about 0.2. Thus, for example, when the predetermined value is set to 0.2, the number of training images 32 that constitute the training image group 32G is two. In this case, presumably, the first object distance is an object distance outside the depth of field, and the second object distance is an object distance inside the depth of field.

Further, it is desirable that the predetermined value is equal to or less than 0.1. In other words, in the information processing system 100 according to the present embodiment, the predetermined value is set to be equal to or less than 0.1. Further, it is desirable that the predetermined value is equal to or less than 0.05. In other words, in the information processing system 100 according to the present embodiment, the predetermined value is set to be equal to or less than 0.05. In this way, the number of training images 32 that constitute the training image group 32G can be increased. With this configuration, when a processing target image captured at an object distance other than an object distance not used in machine learning is input, the trained model 120 is more likely to output a corrected image from which the effect of a blur is appropriately removed. In other words, the accuracy of the correction processing (step S30) by the trained model 120 can be improved more. If the number of training images 32 that constitute the training image group 32G increases, the processing load of machine learning increases. Thus, an appropriate number of training images 32 that constitute the training image group 32G is determined as appropriate depending on a situation.

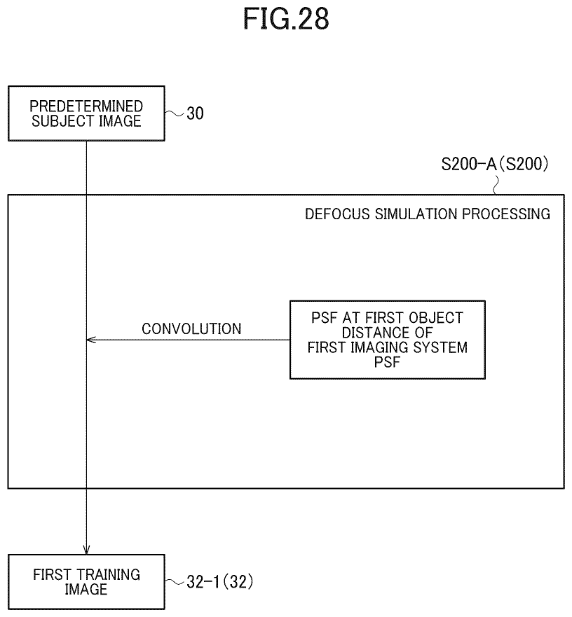

A specific technique for the training device processing section 16 to perform the defocus simulation processing (step S200) and the like using the point spread function will now be described. For example, in a case where the first training image 32-1 is generated by step S200-1, as illustrated in FIG. 28, the training device processing section 16 performs convolution computation processing for the predetermined subject image 30 using the PSF at the first object distance of the first imaging system 101. The convolution can also be referred to as convolution integral. The PSF at the first object distance here is the PSF of the region indicated by FC22-1 in FIG. 12. In other words, in the case of the technique in FIG. 28, the convolution computation processing of the PSF corresponds to step S210 in FIG. 13. Similarly, in a case where the Nth training image 32-N is generated by step S200-N, the training device processing section 16 performs convolution computation processing for the predetermined subject image 30 using the PSF at the Nth object distance of the first imaging system 101. The defocus simulation processing based on the convolution computation processing of the PSF can be called step S200-A. Based on the foregoing, in the information processing system 100 according to the present embodiment, the defocus simulation processing (step S200) is processing of performing convolution computation of the PSF at each of object distances of the first imaging system 101 for the predetermined subject image 30. In this way, the trained model 120 trained by machine learning with a data set of the training images 32 using the PSF and the true image 36 can be generated.

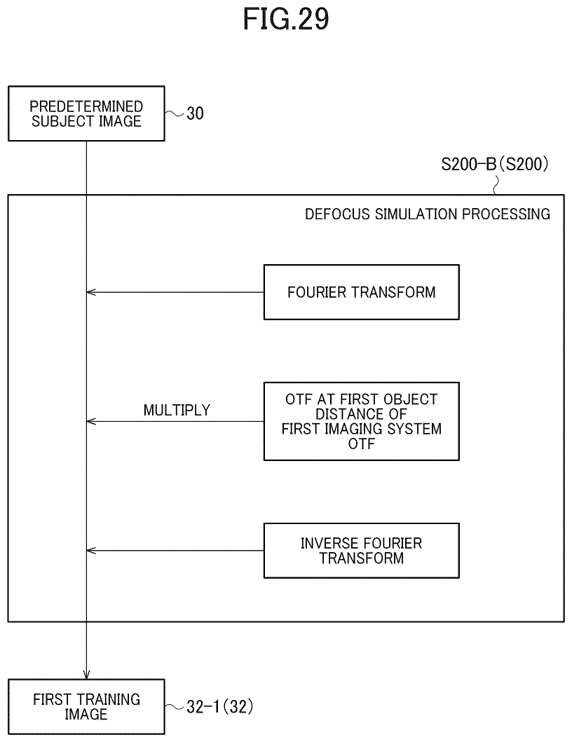

A specific technique for the training device processing section 16 to perform the defocus simulation processing (step S200) using the transfer function will now be described. For example, in a case where the first training image 32-1 is generated, as illustrated in FIG. 29, the training device processing section 16 performs processing of performing Fourier transform of the predetermined subject image 30, processing of multiplying a frequency characteristic which is the result of the Fourier transform by the OTF at the first object distance of the first imaging system 101, and processing of performing inverse Fourier transform of the multiplied frequency characteristic. The OTF at the first object distance here is the OTF of the region indicated by FC22-1 in FIG. 12. In other words, in the case of the technique in FIG. 29, the multiplication by the OTF corresponds to step S210 in FIG. 13. Similarly, in a case where the Nth training image 32-N is generated by step S200-N, the training device processing section 16 performs processing of performing Fourier transform of the predetermined subject image 30, processing of multiplying a frequency characteristic which is the result of the Fourier transform by the OTF at the Nth object distance of the first imaging system 101, and processing of performing inverse Fourier transform of the multiplied frequency characteristic. The defocus simulation processing based on the multiplication by the OTF can be called step S200-B. Based on the foregoing, in the information processing system 100 according to the present embodiment, the defocus simulation processing (step S200) is processing of performing Fourier transform of the predetermined subject image 30, multiplying the frequency characteristic of the predetermined subject image 30 which is the result of the Fourier transform by the OTF at each object distance of the first imaging system 101, and performing inverse Fourier transform of the multiplied frequency characteristic. In this way, the trained model 120 trained by machine learning with a data set of the training images 32 using the OTF and the true image 36 can be generated.

Since the relationship between PSF and OTF is as described above, the computation processing result for the processing in FIG. 28 and the computation processing result for the processing in FIG. 29 are mathematically equivalent. Which of PSF and OTF is to be used in the defocus simulation processing (step S200) may be selected as appropriate by the user.

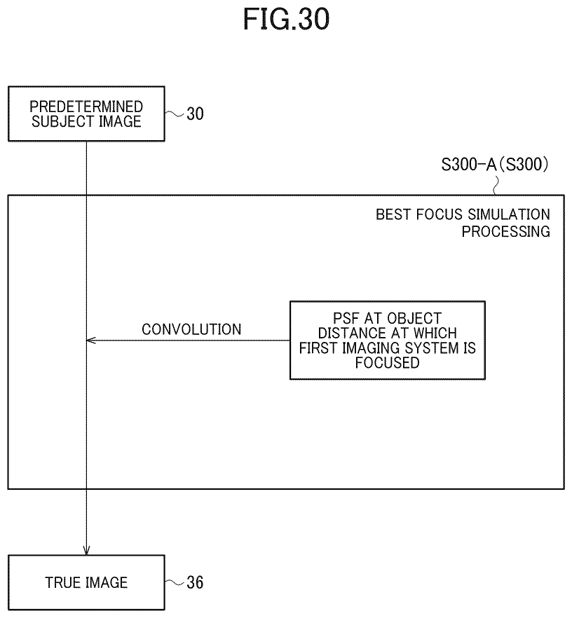

Similarly, the training device processing section 16 may perform the best focus simulation processing (step S300) using the point spread function. For example, as illustrated in FIG. 30, the training device processing section 16 generates the true image 36 by performing convolution computation processing for the predetermined subject image 30, using the PSF at the object distance at which the first imaging system 101 is focused. The best focus simulation processing based on the convolution computation processing of the PSF can also be called step S300-A.

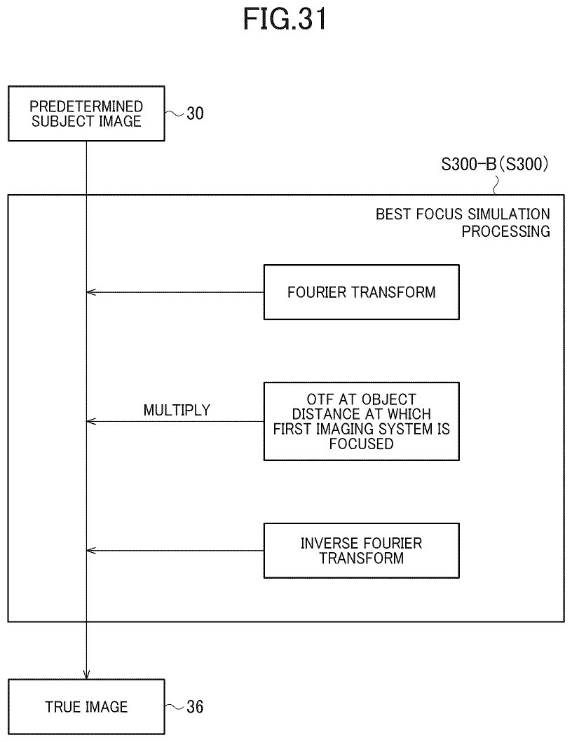

Further, the training device processing section 16 may perform the best focus simulation processing (step S300) using the transfer function. For example, as illustrated in FIG. 31, the training device processing section 16 generates the true image 36 by performing processing of performing Fourier transform of the predetermined subject image 30, processing of multiplying a frequency characteristic which is the result of the Fourier transform by the OTF at the object distance at which the first imaging system 101 is focused, and processing of performing inverse Fourier transform of the multiplied frequency characteristic. The best focus simulation processing based on the multiplication by the OTF can also be called step S300-B.

In the following description, an example in which the training image 32 and the true image 36 are generated using the technique using the PSF will be described, but it is not intended to preclude the technique using the OTF from being applied.

For example, the first imaging system 101 in the present embodiment may have a retrofocus lens configuration. The retrofocus configuration is also called inverted telephoto configuration. For example, the retrofocus lens configuration can be implemented, for example, by disposing a lens with a negative refractive power and a lens with a positive refractive power from the subject side. In the following description, a lens group on the subject side is referred to as front lens group, and a lens group on the image side is referred to as rear lens group.

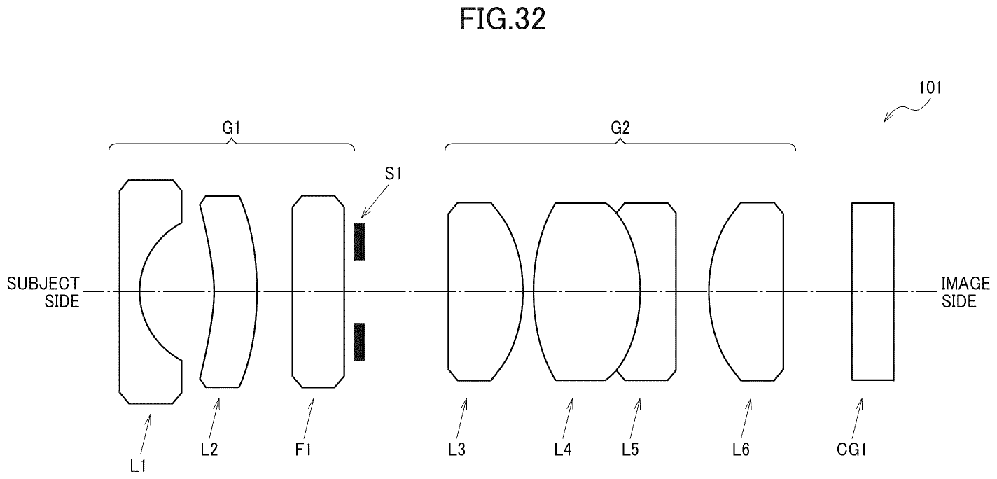

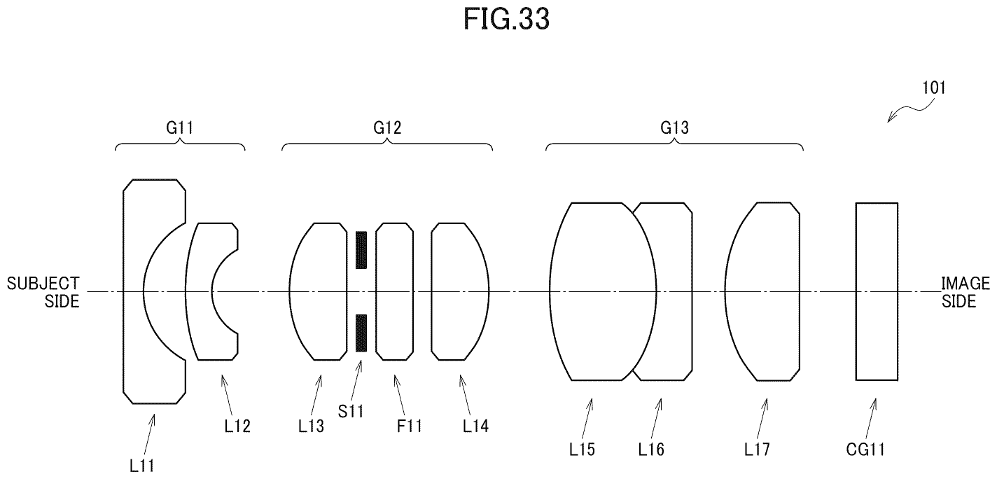

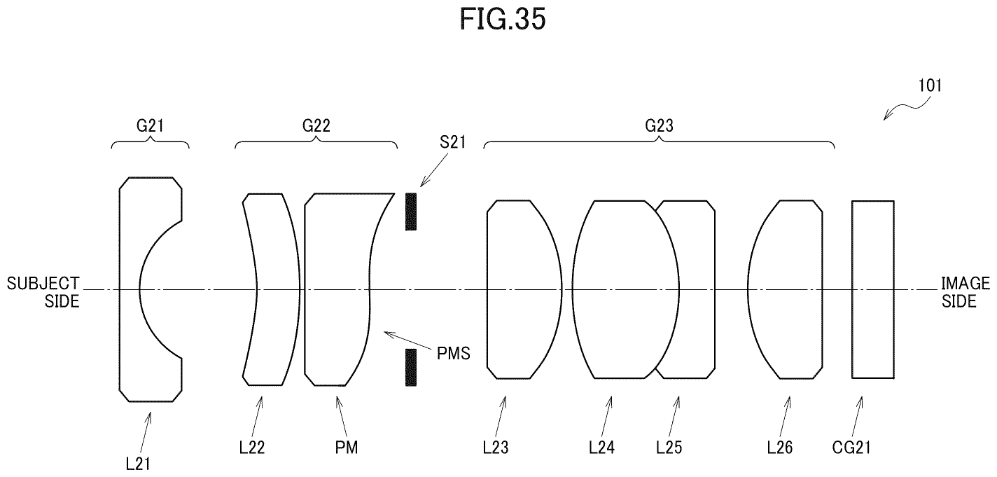

Various known configurations can be employed as a specific retrofocus lens configuration. For example, an optical system illustrated in FIG. 32 includes, in order from the subject side, a front lens group indicated by G1, an aperture stop indicated by S1, a rear lens group indicated by G2, and a cover glass indicated by CG1. In FIG. 32, a space between lenses included in the optical system is not accurately illustrated for convenience of explanation. For example, in FIG. 32, a positive lens indicated by L6 and the cover glass indicated by CG1 are actually cemented but are depicted as being spaced apart from each other for the sake of convenience. This is applicable to FIG. 33 and FIG. 35 described later.

In FIG. 32, the front lens group indicated by G1 includes an objective-side negative lens indicated by L1 and a positive lens indicated by L2 and has a negative refractive power as a whole. The rear lens group indicated by G2 includes a positive lens indicated by L3, a cemented lens including a positive lens indicated by L4 and a negative lens indicated by L5, and a positive lens indicated by L6, and has a positive refractive power as a whole.

The front lens group or the rear lens group may include a plurality of lens groups. For example, in the first imaging system 101 illustrated in FIG. 33, a lens group indicated by G11 functions as a front lens group, and a lens group indicated by G12 and a lens group indicated by G13 function as a rear lens group. For example, the lens group indicated by G11 includes, in order from the subject side, a planoconcave lens having a concave surface facing the image side as indicated by L11 and a negative meniscus lens as indicated by L12 and has a negative refractive power as a whole.

For example, the lens group indicated by G12 includes a subject-side positive lens indicated by L13 and an image-side positive lens indicated by L14. An aperture stop indicated by S11 may be further disposed between the lens indicated by L13 and the lens indicated by L14. In this way, the optical system is configured such that the refractive power is symmetric with respect to the aperture stop, thereby favorably correcting coma and astigmatism.

The lens group indicated by G13 has a positive refractive power as a whole. Further, the lens group indicated by G13 may include a cemented lens including a positive lens indicated by L15 and a negative lens indicated by L16. This configuration can favorably correct spherical aberration and coma. Further, the lens group indicated by G13 may further include a planoconvex lens indicated by L17. This configuration can ensure a wide field of view. The planoconvex lens indicated by L17 and the cover glass indicated by CG11 are depicted as being spaced apart from each other in FIG. 33 but actually they are cemented. The cover glass indicated by CG11 is provided in a not-illustrated image sensor, and the planoconvex lens indicated by L17 is used for positioning of the image sensor.

Further, for example, the first imaging system 101 may further include a parallel plate. The parallel plate is also called a filter. The parallel plate is disposed, for example, at a position F1 in FIG. 32 or a position F11 in FIG. 33 but may be disposed at another position. The parallel plate is used, for example, for the purpose of adjusting the position of image point.

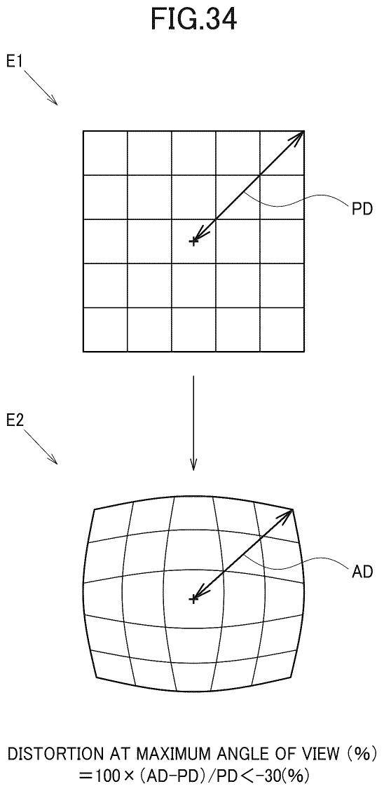

In the first imaging system 101 including the retrofocus lens configuration as described above, it is desirable that the amount of distortion at a maximum angle of view is equal to or less than −30%. Specifically, for example, it is assumed that an image of a subject indicated by E1 in FIG. 34 is captured as an image indicated by E2 in FIG. 34 by the first imaging system 101. In this case, the value of the amount of distortion (%) at the maximum angle of view can be expressed by (AD−PD)/PD×100 using a length indicated by PD of the subject indicated by E1 and a length indicated by AD of the image indicated by E2. It is desirable that the value is a negative value smaller than −30. Based on the foregoing, in the information processing system 100 according to the present embodiment, the first imaging system 101 has a retrofocus lens configuration and the amount of distortion at the maximum angle of view is equal to or less than −30%. In this way, the magnification in a periphery is smaller than that of an image center, so that the transfer function or the point spread function of a region other than on the optical axis can be reduced. Further, the difference between the transfer function or the point spread function of the region on the optical axis and the transfer function or the point spread function of a region other than on the optical axis can be reduced. As a result, the training image 32 with a more accurate result of simulation of the effect of a blur can be generated.

The front lens group or the rear lens group may be configured with a single lens. For example, the first imaging system 101 illustrated in FIG. 35 includes a lens group indicated by G21, a lens group indicated by G22, an aperture stop indicated by S21, a lens group indicated by G23, and a cover glass indicated by CG21. The lens group indicated by G21 includes a single negative lens indicated by L21 and has a negative refractive power. In other words, the lens group indicated by G21 functions as a part of the front lens group. The lens group indicated by G23 includes a positive lens indicated by L23, a cemented lens including a positive lens indicated by L24 and a negative lens indicated by L25, and a positive lens indicated by L26, and has a positive refractive power as a whole. In other words, the lens group indicated by G23 functions as a rear lens group.

Further, the first imaging system 101 in the present embodiment may further include a phase modulation element. For example, a second lens group G2 in FIG. 35 includes a positive lens indicated by L22, an aperture stop indicated by S21, and a phase modulation element indicated by PM. The phase modulation element indicated by PM is disposed at the pupil of the first imaging system 101. The phase modulation element indicated by PM is an element that employs wavefront coding (WFC) and, for example, has a phase modulation surface indicated by PMS. The wave coding is a known technique used in extended depth of field (EDOF) and will not be further elaborated here.

In FIG. 35, the phase modulation surface indicated by PMS is illustrated so as to be represented by a predetermined cubic function using a coordinate orthogonal to the optical axis, but the surface shape of the phase modulation surface is not limited to this and another surface shape may be employed. Further, in FIG. 35, the phase modulation surface is illustrated on the image side but can be disposed on the subject side to achieve a similar effect. Further, the lens group indicated by G22 has a positive refractive power as a whole and also functions as a part of a retrofocus front lens group.

Further, with the inclusion of the phase modulation element indicated by PM, the MTF of the first imaging system 101 less changes with defocus. In other words, the inclusion of the phase modulation element acts such that the MTF of the first imaging system 101 matches with a change in object distance. More specifically, for example, the difference between the MTF of the first object distance and the MTF of the second object distance in the first imaging system 101 that includes the phase modulation element is smaller than the difference between the MTF of the first object distance and the MTF of the second object distance in the first imaging system 101 that does not include the phase modulation element.

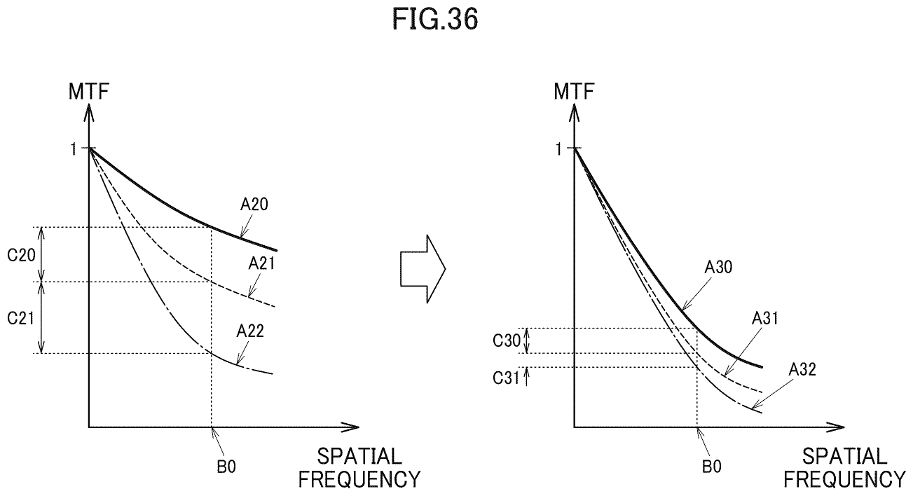

For example, in a relationship between MTF and spatial frequency illustrated in FIG. 36, it is assumed that A20 is the MTF of the first imaging system 101 at an object distance that achieves focus, A21 is the MTF at an object distance with a larger amount of defocus than that of the object distance associated with A20, and A22 is the MTF at an object distance with a larger amount of defocus than that of the object distance associated with A21. Further, it is assumed that A20 to A22 are the MTF of the first imaging system 101 that does not include the phase modulation element. When the aforementioned predetermined spatial frequency indicated by B0 is determined, the difference between the MTF of A20 and the MTF of A21 is a difference indicated by C20, and the difference between the MTF of A21 and the MTF of A22 is a difference indicated by C21. In FIG. 36, the MTF in a frequency higher than the spatial frequency indicated by B0 is partially not illustrated.

Here, since the phase modulation element indicated by PM is included in the first imaging system 101, the MTF indicated by A20 changes to the MTF indicated by A30, the MTF indicated by A21 changes to the MTF indicated by A31, and the MTF indicated by A22 changes to the MTF indicated by A32. Further, the difference in MTF indicated by C20 is reduced as indicated by C30, and the difference in MTF indicated by C21 is reduced as indicated by C31. Based on the foregoing, in the information processing system 100 according to the present embodiment, the first imaging system 101 further includes an optical wavefront modulation element that changes the transfer function or the point spread function. In this way, the distance necessary for machine learning can be reduced, so that the number of data sets necessary for machine learning can be reduced.

The example of the defocus simulation processing (step S200) and the like described above is a processing example for generating the training image 32 based on optical information of the first imaging system 101 for the predetermined subject image 30 captured by the given imaging system 104. The technique of the present embodiment is not limited thereto. For example, the training device processing section 16 may perform the defocus simulation processing so as to further include processing that simulates removal of the effect of imaging by the given imaging system 104 from the predetermined subject image 30.

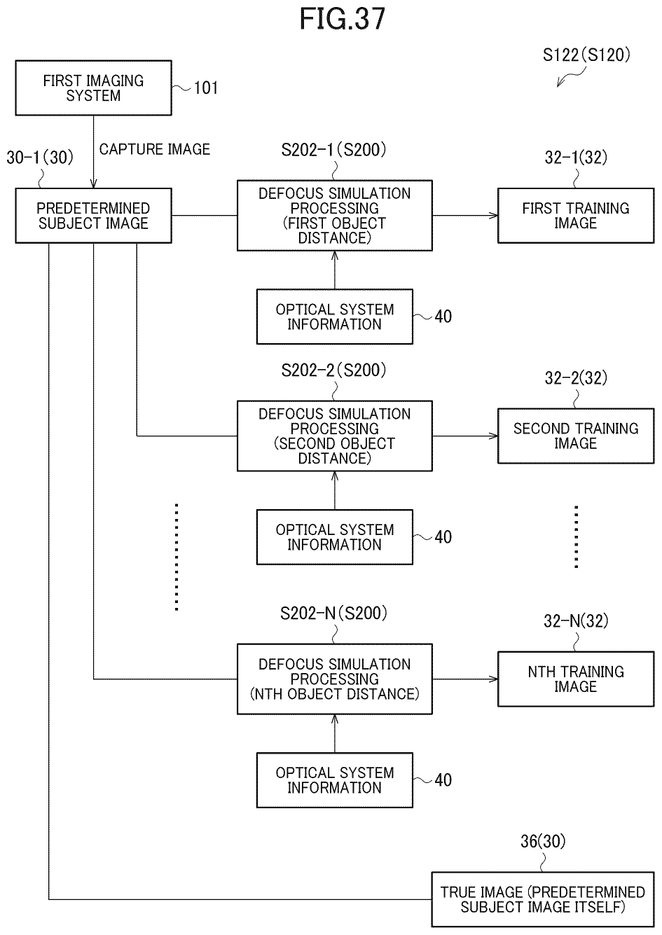

FIG. 37 illustrates an example of the image data generation processing in a case of further including the processing that simulates, for a predetermined subject image 30-1 captured by the first imaging system 101, removal of the effect of imaging by removal of the effect of the first imaging system 101. The image data generation processing illustrated in FIG. 37 can also be called step S122. Step S122 in FIG. 37 differs from step S120-2 in FIG. 11 in the content of the defocus simulation processing. FIG. 37 is common to FIG. 11 in that the best focus simulation processing (step S300) is not performed and the true image 36 is the predetermined subject image 30-1 itself. This is because the predetermined subject image 30-1 is an image captured under the best focus condition of the first imaging system 101 and there is no need for performing processing similar to step S202 in the first place.

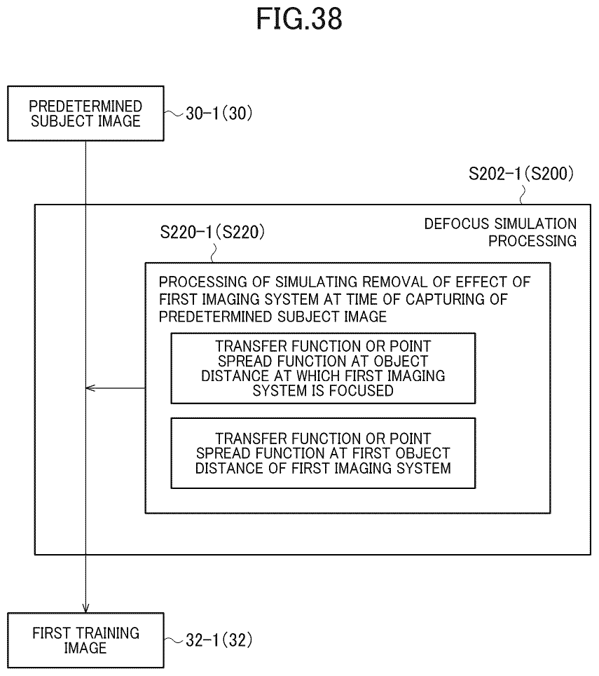

FIG. 38 illustrates an example of defocus simulation processing (step S202-1) in the image data generation processing (step S122). For example, in a case where the first training image 32-1 is generated, the training device processing section 16 performs the processing of simulating, for the predetermined subject image 30-1, removal of the effect of the first imaging system 101 at the time of capturing the predetermined subject image 30-1 (step S220-1). Step S220-1 is performed based on the transfer function or the point spread function at the object distance at which the first imaging system 101 is focused, and the transfer function or the point spread function at the first object distance of the first imaging system 101.

More specifically, for example, the training device processing section 16 performs, for the predetermined subject image 30, computation processing that appropriately combines computation processing of performing deconvolution of the PSF at the object distance at which the first imaging system 101 is focused and computation processing of performing convolution of the PSF at the first object distance of the first imaging system 101 (step S200-A). The “computation processing that appropriately combines” refers to computation processing in which one computation processing is combined with part or the whole of the other computation processing in a given order, but it is not intended to preclude one computation processing and the other computation processing from being performed separately. The computation processing that appropriately combines is determined as appropriate depending on a predetermined situation. This is applicable in the following description. The predetermined situation is, for example, the processing time required for machine learning, the processing load on processors, and the like. In other words, step S220-1 can be performed to obtain, for example, a computation processing result that reflects both of the effect of the computation processing of performing deconvolution of the PSF at the object distance at which the first imaging system 101 is focused and the effect of the computation processing of performing convolution of the PSF at the first object distance of the first imaging system 101 (step S200-A), for the predetermined subject image 30-1.

Based on the foregoing, in the information processing system 100 according to the present embodiment, the given imaging system 104 is the first imaging system 101. The defocus simulation processing (step S202) further includes processing of removing the effect of the first imaging system 101 from the predetermined subject image 30-1, based on the transfer function or the point spread function at the object distance at which the first imaging system 101 is focused, and the transfer function or the point spread function at a plurality of object distances of the first imaging system 101 (step S212). In this way, a more accurate training image 32 can be generated. The training image 32 and the true image 36 according to the technique illustrated in FIG. 10 and FIG. 11 have both the effect of the given imaging system 104 and the effect of the first imaging system 101 on a predetermined subject, whereas the training image 32 and the true image 36 according to the technique illustrated in FIG. 37 and FIG. 38 have only the effect of the first imaging system 101 on a predetermined subject. With this configuration, machine learning with more appropriate data sets can be performed.

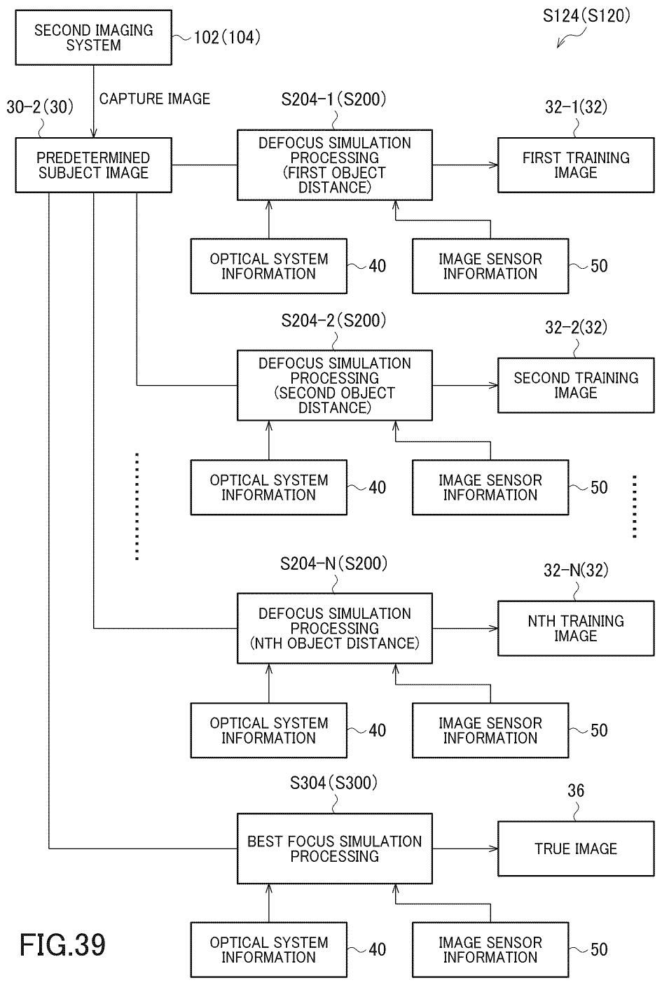

Similarly, FIG. 39 illustrates an example of the image data generation processing including the processing that simulates removal of the effect of imaging by the given imaging system 104. In FIG. 39, a second imaging system 102 is illustrated as a representative of the given imaging system 104. Further, it is assumed that the second imaging system 102 is an imaging system with an image sensor with a higher resolution, compared with the first imaging system 101. Further, the image data generation processing illustrated in FIG. 39 can also be called step S124, and an original image for step S124 can also be called a predetermined subject image 30-2.

Step S126 in FIG. 39 differs from step S120-1 in FIG. 10 in that the defocus simulation processing (step S204) and the best focus simulation processing (step S304) are performed after image sensor information 50 is further read. The image sensor information 50 is information regarding a resolution of an image sensor included in each of the first imaging system 101 and the given imaging system 104. In other words, in a case of the example in FIG. 39, the image sensor information 50, which is not depicted in FIG. 4, is further stored in the training device memory section 18. The image sensor information 50 is also used in computation processing in the defocus simulation processing (step S204) and the best focus simulation processing (step S304).

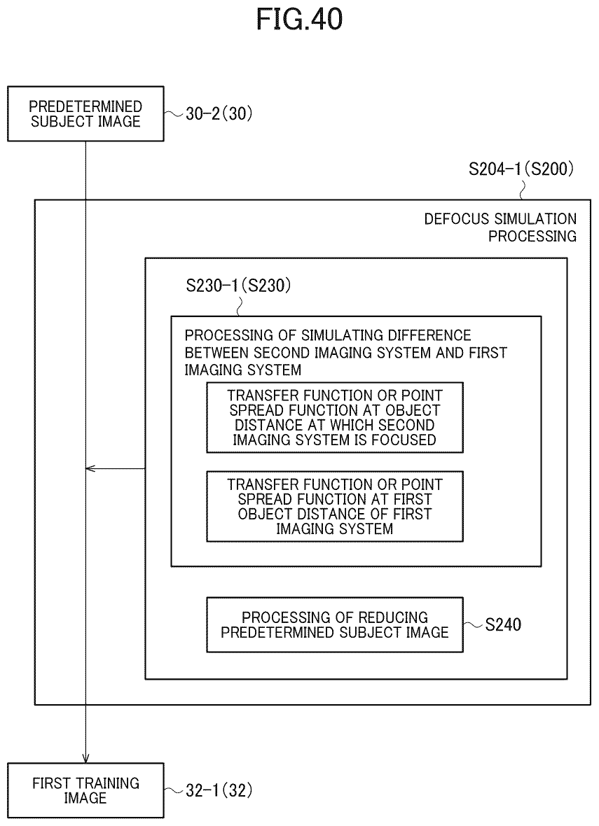

FIG. 40 illustrates an example of the defocus simulation processing in the image data generation processing (step S124) illustrated in FIG. 39. The defocus simulation processing illustrated in FIG. 39 and FIG. 40 can also be called step S204. For example, in a case where the first training image 32-1 is generated, the training device processing section 16 performs, for the predetermined subject image 30-2, computation processing that appropriately combines processing of simulating the difference between the second imaging system 102 and the first imaging system 101 (step S230-1), processing of reducing the predetermined subject image 30 (step S240), and computation processing based on the image sensor information 50 not depicted in FIG. 40. Step S230-1 is performed based on the transfer function or the point spread function at an object distance at which the second imaging system 102 is focused, and the transfer function or the point spread function at the first object distance of the first imaging system 101. In other words, step S230-1 can be performed to obtain a computation processing result that reflects both of the effect of the computation processing of performing deconvolution of the PSF at the object distance at which the second imaging system 102 is focused and the effect of the computation processing of performing convolution of the PSF at the first object distance of the first imaging system 101 (step S200-A), for the predetermined subject image 30-2. Further, step S204-1 can be performed to obtain a computation processing result that reflects the effect of the computation processing in step S230-1, the effect of the computation processing in step S240, and the effect of the computation processing based on the image sensor information 50.

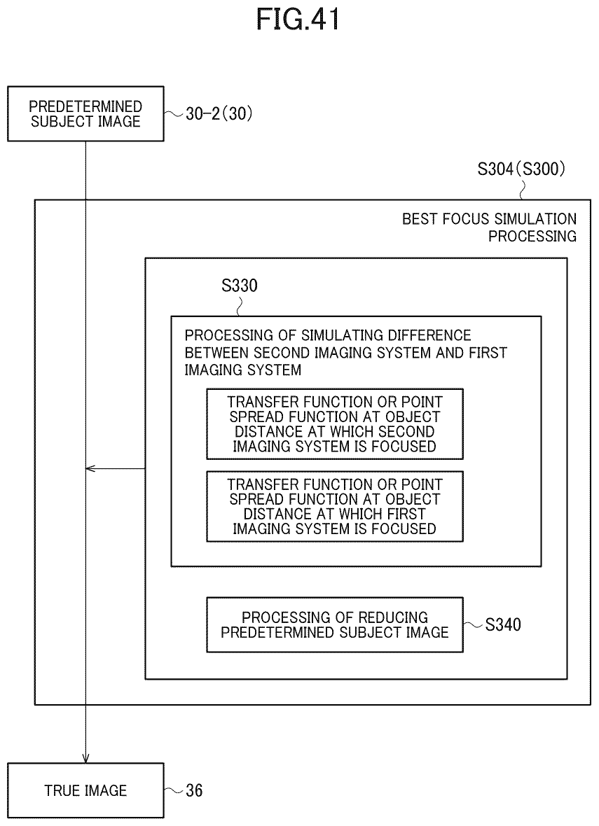

FIG. 41 illustrates an example of the best focus simulation processing illustrated in FIG. 39. The best focus simulation processing illustrated in FIG. 39 and FIG. 41 can also be called step S304. For example, the training device processing section 16 performs, for the predetermined subject image 30-2, processing that appropriately combines processing of simulating the difference between the second imaging system 102 and the first imaging system 101 (step S330), processing of reducing the predetermined subject image 30-2 (step S340), and computation processing based on the image sensor information 50 not depicted in FIG. 41. As a result, the training device processing section 16 can generate the true image 36. Step S330 in FIG. 41 is performed based on the transfer function or the point spread function at the object distance at which the second imaging system 102 is focused, and the transfer function or the point spread function at the object distance at which the first imaging system 101 is focused. In other words, step S330 can be performed to obtain, for example, a computation processing result that reflects both of the effect of the computation processing of performing deconvolution of the PSF at the object distance at which the second imaging system 102 is focused and the effect of the computation processing of performing convolution of the PSF at the distance at which the first imaging system 101 is focused (step S300-A), for the predetermined subject image 30-2. Further, step S340 in FIG. 41 is computation processing similar to step S240 in FIG. 40. Further, step S304 can be performed to obtain a computation processing result that reflects the effect of the computation processing in step S330, the effect of the computation processing in step S340, and the effect of the computation processing based on the image sensor information 50. The true image 36 may be generated by processing in which step S330 is eliminated from the best focus simulation processing (step S304) in FIG. 41. In other words, the true image 36 may be generated by performing processing corresponding to step S340 for the predetermined subject image 30-2. This is because if the predetermined subject image 30-2 is an image captured at the object distance at which the given imaging system 104 is focused, the true image 36 can be obtained by changing the number of pixels of the predetermined subject image 30-2 in step S340.

Based on the foregoing, in the information processing system 100 according to the present embodiment, the defocus simulation processing (step S204) further includes processing of simulating the difference between the given imaging system 104 and the first imaging system 101 (step S230) and processing of reducing the predetermined subject image 30-2 (step S240). The true image 36 is an image generated by performing the best focus simulation processing (step S304) or an image generated by performing the processing that reduces the predetermined subject image 30-2. The processing of simulating the difference between the given imaging system 104 and the first imaging system 101 (step S230) in the defocus simulation processing (step S204) is based on the transfer function or the point spread function at the object distance at which the given imaging system 104 is focused, and the transfer function or the point spread function at a plurality of object distances of the first imaging system 101. The best focus simulation processing (step S304) further includes processing of simulating the difference between the given imaging system 104 and the first imaging system 101 (step S330) and processing of reducing the predetermined subject image 30-2 (step S340). The processing of simulating the difference between the given imaging system 104 and the first imaging system 101 (step S330) in the best focus simulation processing (step S304) is based on the transfer function or the point spread function at the object distance at which the given imaging system 104 is focused, and the transfer function or the point spread function at the object distance at which the first imaging system 101 is focused.

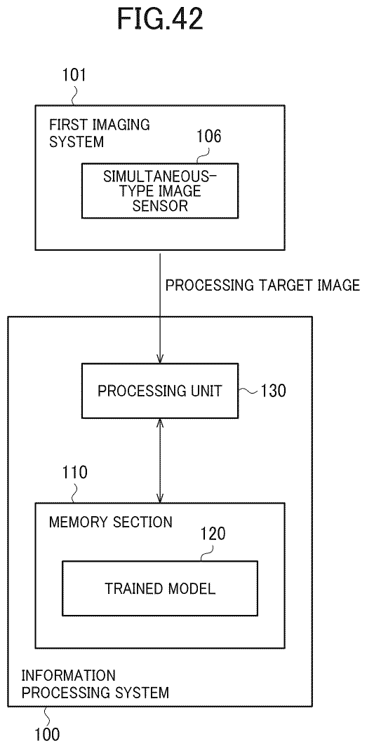

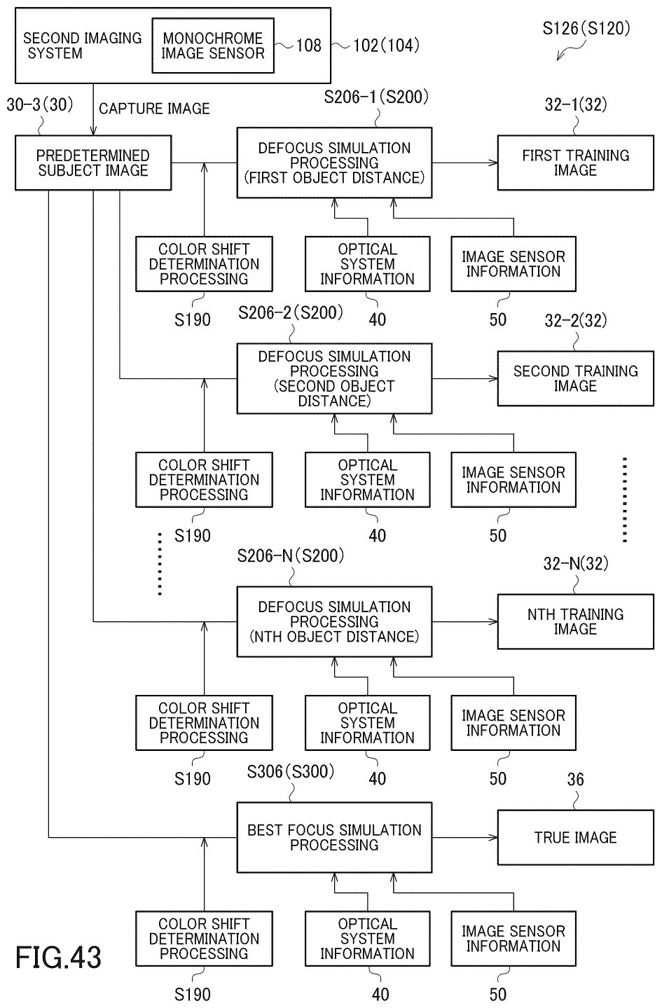

Further, the technique of the present embodiment can also be applied to a case where the given imaging system 104 and the first imaging system 101 employ different imaging methods. For example, as illustrated in FIG. 42, it is assumed that the first imaging system 101 includes a simultaneous-type image sensor 106. Further, as illustrated in FIG. 43, it is assumed that the given imaging system 104 includes a monochrome image sensor 108. Referring to FIG. 43, a technique of image data generation processing in this case will be described. The image data generation processing in FIG. 43 can also be called step S126, and an original image for step S126 can also be called a predetermined subject image 30-3. FIG. 43 differs from FIG. 39 in the contents of the defocus simulation processing (step S206) and the best focus simulation processing (step S306) and in that color shift determination processing (step S190) is performed before steps S206 and S306 are performed. In FIG. 43, the second imaging system 102 is illustrated as a representative of the given imaging system 104, which is the same as in the example in FIG. 39. Further, the color shift determination processing (S190) is, for example, processing of comparing a coloring amount in a periphery of a saturated portion or the like in the predetermined subject image 30-3 with a predetermined threshold. The color shift is a shift that occurs among an R image, a G image, and a B image, for example, due to a difference in imaging timing when an image of a subject is captured using the monochrome image sensor 108. The color shift does not occur in a processing target image captured by the simultaneous-type image sensor 106. Further, the coloring amount in a periphery of a saturated portion or the like in the predetermined subject image 30-3 is a coloring amount that occurs due to the color shift in a periphery of an area appearing white in the predetermined subject image 30-3. In other words, steps S206 and S306 in FIG. 43 use the predetermined subject image 30-3 in which the coloring amount in a periphery of a saturated portion or the like is determined to be equal to or less than a predetermined threshold in step S190. Thus, the training image 32 in which the effect of the color shift is reduced can be generated by performing step S206. Similarly, the true image 36 in which the effect of the color shift is reduced can be generated by performing step S306. With this configuration, in the case where the given imaging system 104 and the first imaging system 101 employ different imaging methods, an appropriate data set including the training images 32 and the true image 36 can be generated.

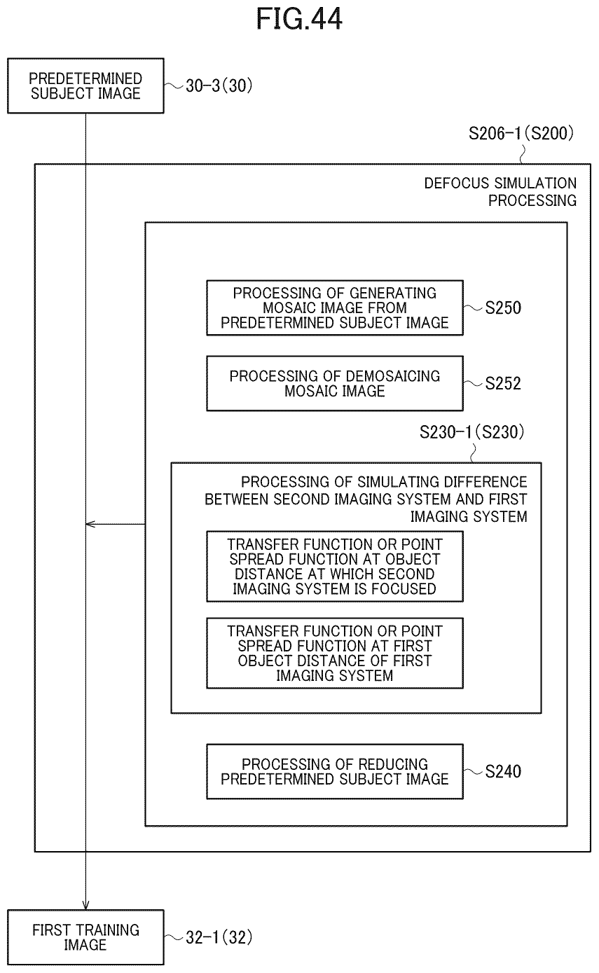

FIG. 44 illustrates an example of the defocus simulation processing in the image data generation processing (step S126) illustrated in FIG. 43. The defocus simulation processing illustrated in FIG. 43 and FIG. 44 can also be called step S206. FIG. 44 differs from FIG. 40 in that it further includes processing of generating a mosaic image from the predetermined subject image 30-3 (step S250) and processing of demosaicing the mosaic image (step S252). For example, in a case where the first training image 32-1 is generated, the training device processing section 16 performs, for the predetermined subject image 30-3, computation processing that appropriately combines the above step S230-1, the above step S240, step S250, step S252, and computation processing based on the image sensor information 50 not depicted in FIG. 44. In other words, step S206-1 can be performed to obtain a computation processing result that reflects the effect of the computation processing in step S230-1, the effect of the computation processing in step S240, the effect of the computation processing in step S250, the effect of the computation processing in step S252, and the effect of the computation processing based on the image sensor information 50.

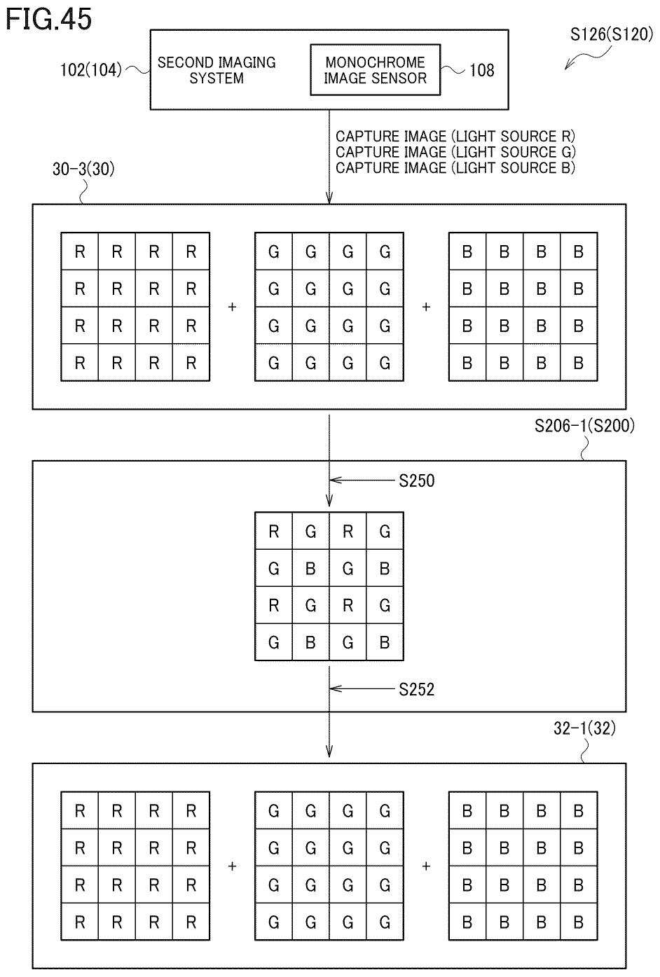

Steps S250 and S252 will now be described specifically. The predetermined subject image 30-3 is a field sequential image obtained by processing of combining a plurality of images captured by the monochrome image sensor 108 at a timing when light of each wavelength band is emitted in a case where light having a plurality of wavelength bands is sequentially emitted. For example, as illustrated in FIG. 45, in step S206-1 described above, processing including step S250 generates a mosaic image. Processing including step S252 generates a field sequential image again from the mosaic image, whereby the first training image 32-1 is generated. In step S206-1 in FIG. 45, processing other than steps S250 and S252 is not depicted.

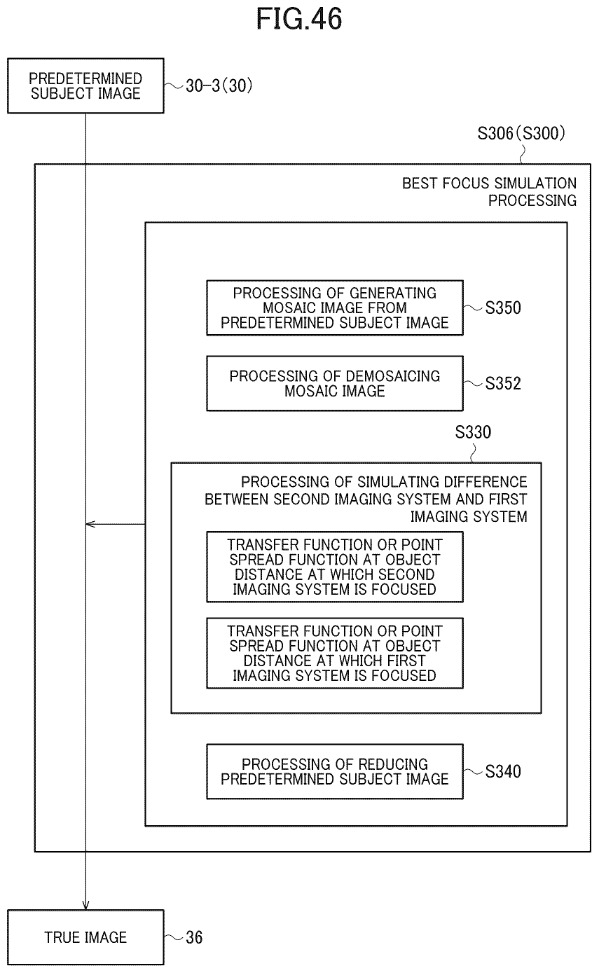

FIG. 46 illustrates an example of the best focus simulation processing in the image data generation processing (step S126) illustrated in FIG. 43. The best focus simulation processing illustrated in FIG. 43 and FIG. 46 can also be called step S306. FIG. 46 differs from FIG. 41 in that it further includes processing of generating a mosaic image from the predetermined subject image 30-3 (step S350) and processing of demosaicing the mosaic image (step S352). Further, step S350 in FIG. 46 is processing similar to step S250 in FIG. 44, and step S352 in FIG. 46 is processing similar to step S252 in FIG. 44. For example, the training device processing section 16 performs computation processing that appropriately combines the above step S330-1, the above step S340, step S350, step S352, and computation processing based on the image sensor information 50 not depicted in FIG. 46. As a result, the training device processing section 16 can generate the true image 36. Thus, step S306 can be performed to obtain a computation processing result that reflects the effect of the computation processing in step S330, the effect of the computation processing in step S340, the effect of the computation processing in step S350, the effect of the computation processing in step S352, and the effect of the computation processing based on the image sensor information 50. The true image 36 may be generated by processing in which steps S330, S350, and S352 are omitted from the best focus simulation processing (step S306). In other words, the true image 36 may be generated by performing processing corresponding to step S340 for the predetermined subject image 30-3.

Based on the foregoing, in the information processing system 100 according to the present embodiment, the given imaging system 104 includes the monochrome image sensor 108. The predetermined subject image 30-3 is a field sequential image obtained by processing of combining a plurality of images captured by the monochrome image sensor 108 at a timing when light of each wavelength band is emitted in a case where light having a plurality of wavelength bands is sequentially emitted. The first imaging system 101 includes the simultaneous-type image sensor 106 that has a plurality of pixels having colors different from each other and in which one color is allocated to each of the pixels. The defocus simulation processing (step S206) further includes processing of generating, from the predetermined subject image 30-3, a mosaic image in which one color is allocated to each of the pixels, processing of demosaicing the mosaic image, processing of simulating the difference between the given imaging system 104 and the first imaging system 101, and processing of reducing the predetermined subject image 30-3. The processing of simulating the difference between the given imaging system 104 and the first imaging system 101 in the defocus simulation processing (step S206) is based on the transfer function or the point spread function at the object distance at which the given imaging system 104 is focused, and the transfer function or the point spread function at a plurality of object distances of the first imaging system 101. The true image 36 is an image generated by performing the best focus simulation processing (step S306) or an image generated by performing the processing of reducing the predetermined subject image 30-3. The best focus simulation processing (step S306) further includes processing of generating a mosaic image, processing of demosaicing the mosaic image, processing of simulating the difference between the given imaging system 104 and the first imaging system 101, and processing of reducing the predetermined subject image 30-3. The processing of simulating the difference between the given imaging system 104 and the first imaging system 101 in the best focus simulation processing (step S306) is based on the transfer function or the point spread function at the object distance at which the given imaging system 104 is focused, and the transfer function or the point spread function at the object distance at which the first imaging system 101 is focused. In this way, in the case where the imaging method of the predetermined subject image 30 and the imaging method of the processing target image are different, a more appropriate data set including the training images 32 and the true image 36 can be generated.

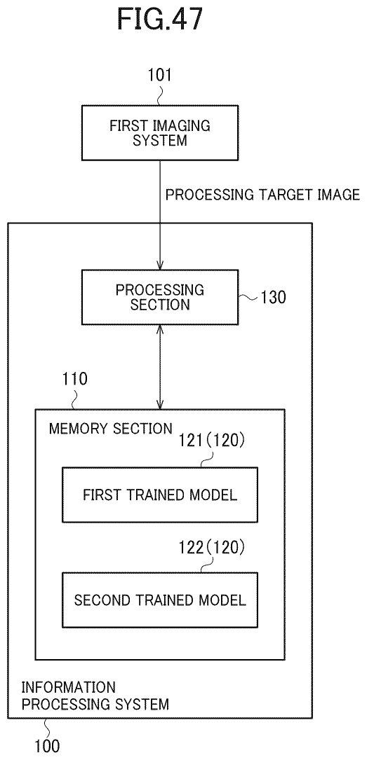

Different trained models 120 may be used depending on imaging methods. In other words, in the information processing system 100 according to the present embodiment, for example, as illustrated in FIG. 47, the memory section 110 may store a first trained model 121 and a second trained model 122.

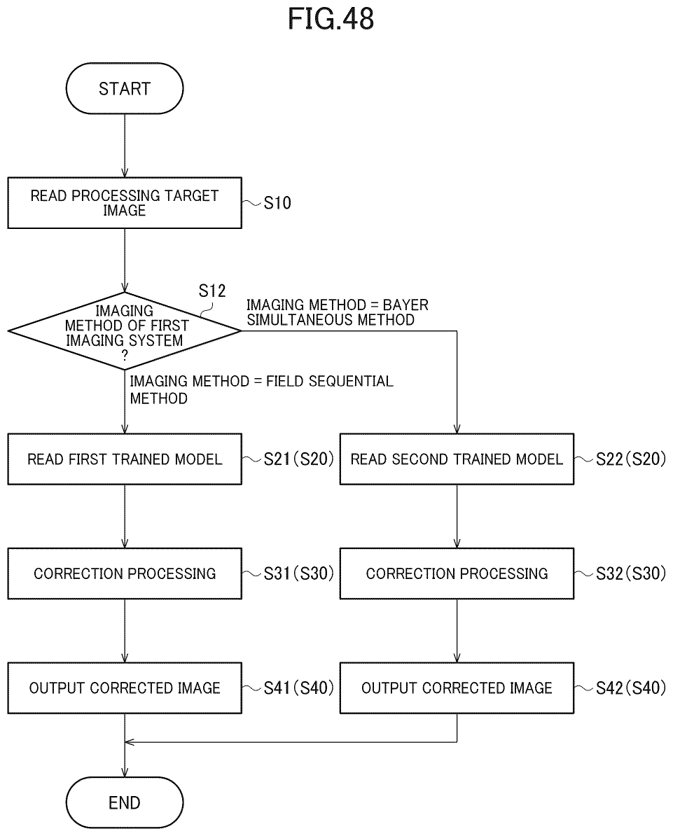

In a case where the memory section 110 stores the first trained model 121 and the second trained model 122, the flow illustrated in FIG. 3 may be replaced, for example, by the flow in FIG. 48. The processing section 130 reads a processing target image (step S10) and thereafter performs processing of checking an imaging method of the first imaging system 101 (step S12). If the imaging method is a field sequential method, reading the first trained model (step S21), correction processing (step S31), and corrected image output (step S41) are performed. On the other hand, if the imaging method is a Bayer simultaneous method, reading the second trained model (step S22), correction processing (step S32), and corrected image output (step S42) are performed. Steps S21 and S22 in FIG. 48 are processing corresponding to step S20 in FIG. 3. Similarly, steps S31 and S32 in FIG. 48 are processing corresponding to step S30 in FIG. 3, and steps S41 and S42 in FIG. 48 are processing corresponding to step S40 in FIG. 3.

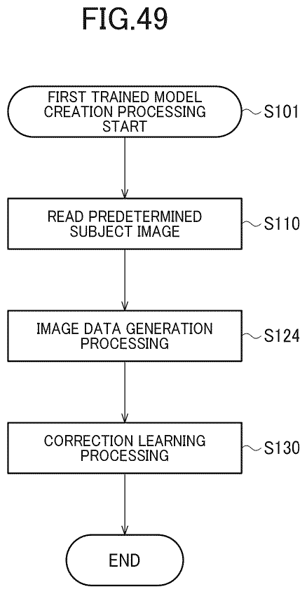

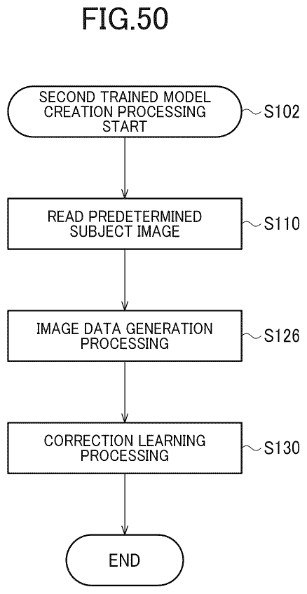

In this case, step S100 in FIG. 7 may be performed as in step S101 in FIG. 49 and step S102 in FIG. 50. Specifically, first trained model creation processing (step S101) in FIG. 49 employs the image data generation processing in step S124 in FIG. 39 for step S100 in FIG. 7. Similarly, second trained model creation processing (step S102) in FIG. 50 employs the image data generation processing in step S126 in FIG. 43 for step S100 in FIG. 7.

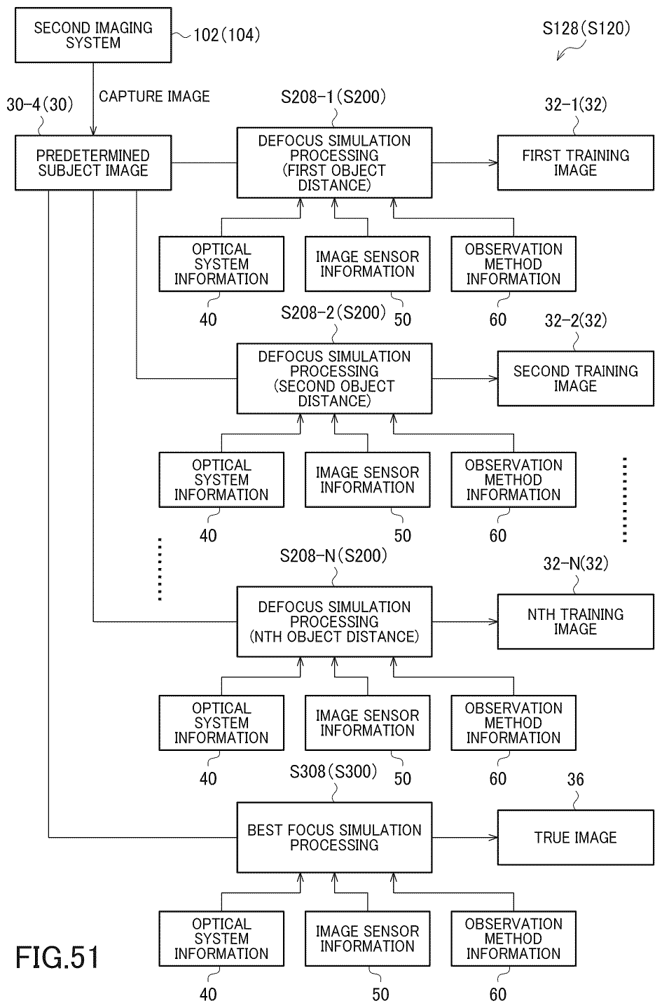

Further, the technique of the present embodiment can also be applied to a case where the given imaging system 104 and the first imaging system 101 employ different observation methods. Referring to FIG. 51, a technique of image data generation processing in the case where observation methods are different will be described. The image data generation processing in FIG. 51 can also be called step S128, and an original image for step S128 can also be called a predetermined subject image 30-4. Step S128 in FIG. 51 differs from step S124 in FIG. 39 in the contents of the defocus simulation processing (step S208) and the best focus simulation processing (step S308) and in that observation method information 60 is read before steps S206 and S306 are performed. The observation method information 60 is, for example, information regarding an observation method in the first imaging system 101. In other words, in a case of the example in FIG. 51, the observation method information 60 not depicted in FIG. 4 is further stored in the training device memory section 18. In FIG. 51, the second imaging system 102 is illustrated as a representative of the given imaging system 104, which is the same as in the example in FIG. 39.

The observation method can also be called an observation mode. The case where observation methods are different may be, for example, a case where light sources for observation are different, but may be, for example, a case where image processing techniques performed from when a user performs processing of capturing an image of a subject to when acquiring the predetermined subject image 30-4 are different. Examples of the observation method include a white light imaging (WLI) mode that uses white illumination light and a special light observation mode that uses special light which is not white light. The special light observation mode includes a narrow band imaging (NBI) mode that uses two types of narrow band light. The two types of narrow band light are narrow band light included in a blue wavelength band and narrow band light included in a green wavelength band. The image processing is different between the WLI and the NBI when a color image is generated from image signals output by the image sensor. For example, a content of the demosaicing processing or a parameter in the image processing is different. As the special light observation mode, for example, a red dichromatic imaging (RDI) mode may be employed. The RDI mode is an observation mode using narrow band light included in an umber wavelength band, narrow band light included in a green wavelength band, and narrow band light included in a red wavelength band. For example, the technique disclosed in U.S. Pat. No. 9,775,497 B2 is used.

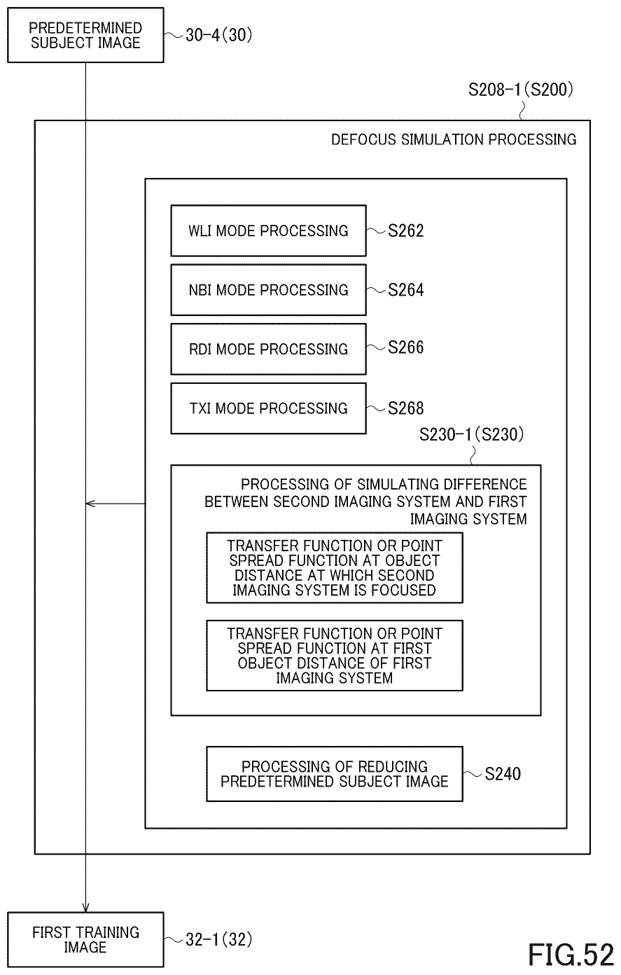

FIG. 52 illustrates an example of defocus simulation processing (step S208-1) of generating the first training image 32-1 from the predetermined subject image 30-4. Step S208-1 in FIG. 52 differs from step S204-1 in FIG. 50 in that it further includes WLI mode processing (step S262), NBI mode processing (step S264), RDI mode processing (step S266), and TXI mode processing (step S268). TXI is an abbreviation of texture and color enhancement imaging, which will be detailed later.

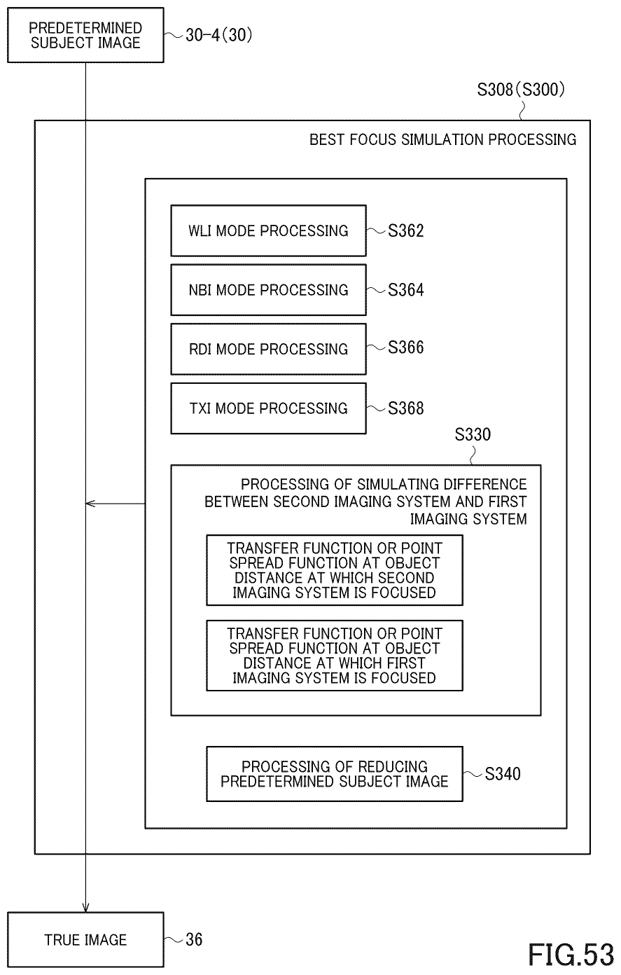

Step S128 in FIG. 51 is an example in which processing and the like that is the different point described above is added to S124 in FIG. 39, but not limited thereto. For example, processing and the like that is the different point described above may be added to step S126 in FIG. 43. In this case, although not depicted in the drawings, the color shift determination processing (step S190) in FIG. 43 is further performed before steps S208 and S308 are performed. Step S208 in FIG. 52 in this case further includes steps S240, S250, and S252 in FIG. 44. Similarly, step S308 in FIG. 53 in this case further includes steps S340, S350, and S352 in FIG. 46. In the following, a description that overlaps with the description of step S124 in FIG. 39 and step S126 in FIG. 43 is omitted if appropriate.

For example, although not illustrated in a flowchart, the training device processing section 16 reads the observation method information 60 and acquires an observation method used for the first imaging system 101. The training device processing section 16 then selects any of steps S262, S264, S266, and S268 as processing corresponding to the acquired observation method.

For example, in a case where the first imaging system 101 captures an image in the TXI mode, information indicating this is stored as observation method information 60 in the training device memory section 18. The training device processing section 16 then reads the observation method information 60 to perform the defocus simulation processing (step S208) including the TXI mode processing (step S368) for the predetermined subject image 30-4. Specifically, for example, the training device processing section 16 performs processing of decomposing the predetermined subject image 30-4 into a texture image portion which is an image portion associated with a surface structure of the predetermined subject image 30-4, and a base image portion other than the texture image portion. The training device processing section 16 then performs first processing of enhancing the surface structure associated with the texture image portion, second processing of optimizing brightness of the base image portion, and third processing of optimizing a color tone of an image that combines an image associated with the first processing and an image associated with the second processing. This can result in the training image 32 that simulates the effect of imaging in the TXI mode for the predetermined subject image 30-4. As a result, machine learning can be performed with a data set including more accurate training images 32.

Further, for example, although not depicted in the drawings, in a case where the first imaging system 101 captures an image in the WLI mode or the NBI mode, information indicating this is stored as observation method information 60 in the training device memory section 18. The training device processing section 16 then reads the observation method information 60 to perform color complementation to match a light source for the predetermined subject image 30-4. The color complementation may be performed, for example, in addition to step S252 in FIG. 44. For example, when the WLI mode processing (step S262) is selected, the training device processing section 16 performs processing of complementing an R image and a B image using a G image, in addition to step S252. For example, when the NBI mode processing (step S264) is selected, the training device processing section 16 performs processing of complementing a G image and a B image independently of each other, in addition to step S252.

FIG. 53 illustrates an example of the best focus simulation processing (step S308) of generating the true image 36 from the predetermined subject image 30-4 in the image data generation processing (step S128). Step S308 in FIG. 53 differs from step S304 in FIG. 41 in that it further includes WLI mode processing (step S362), NBI mode processing (step S364), RDI mode processing (step S366), and TXI mode processing (step S368). Step S362 in FIG. 53 is processing similar to step S262 in FIG. 52, step S364 in FIG. 53 is processing similar to step S264 in FIG. 52, step S366 in FIG. 53 is processing similar to step S266 in FIG. 52, and step S368 in FIG. 53 is processing similar to step S268 in FIG. 52. The true image 36 may be generated by processing in which step S330 or the like is omitted from the best focus simulation processing (step S308) in FIG. 53.

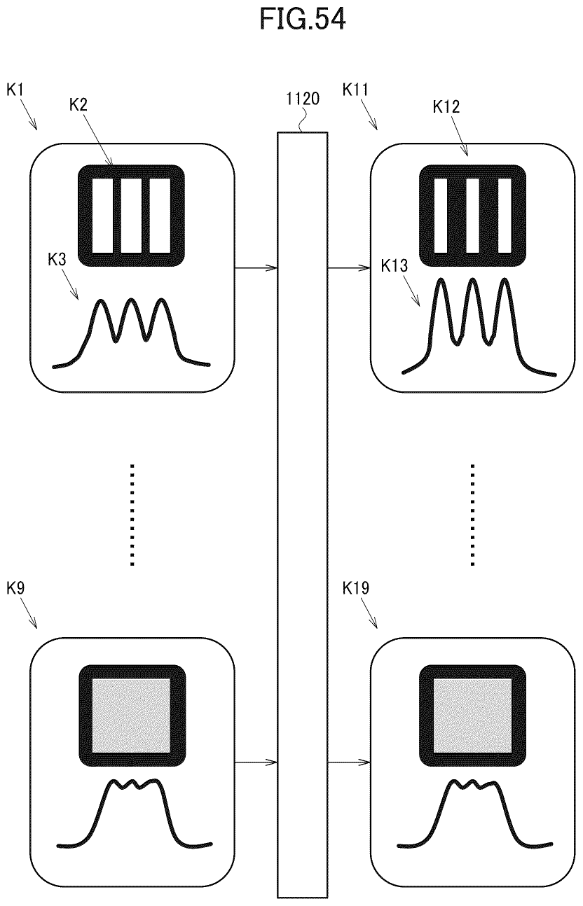

Further, the correction table 1400 in FIG. 25 can be created, for example, based on the result of evaluation of frequency characteristic for each object distance for contrast or amplitude of various image data. For example, in FIG. 54, input data indicated by K1 is input to the trained model 1120, and output data indicated by K11 is output from the trained model 1120. Similarly, input data indicated by K9 is input to the trained model 1120, and output data indicated by K19 is output from the trained model 1120.

Although the image data associated with the input data is an image of a chart denoted by K2 in FIG. 54, it may be an image of biological tissue or an image of a subject that imitates biological tissue as described above, or any image that is equivalent to the training image 32. The contrast of the image of the chart may be common to the contrast of biological tissue as described above. The input data in FIG. 54 may be an actual image of the chart or an image created by simulation. Further, K3 in FIG. 54 is a conceptual representation of the spread function corresponding to the chart indicated by K2. In output data indicated by K11, the chart image indicated by K12 corresponds to the output image 134, and K13 is a conceptual representation of the spread function corresponding to the chart indicated by K12.

In FIG. 54, the input data on the upper side of the drawing sheet indicates that it includes a chart image with lower frequencies, and the input data on the lower side of the drawing sheet indicates that it includes a chart image with higher frequencies. For example, the frequency of the image of the chart indicated by K2 associated with the input data of K1 is lower than the frequency of the image of the chart associated with the input data of K9. Due to the effect of the correction processing (step S1030), the image of the chart associated with the output data of K11 is sharper than the image of the chart indicated by K2. Therefore, the user can quantitatively evaluate the difference in contrast between the training image 32 and the output image 134, or the difference in amplitude between the training image 32 and the output image 134, by analyzing the spread function indicated by K13 and the spread function indicated by K3.

On the other hand, for example, when the input data indicated by K9 in FIG. 54 is compared with the output data indicated by K19, the change in contrast or the change in amplitude by the correction processing (step S1030) is extremely small. The frequency region associated with the input data indicated by K9 in FIG. 54 is, for example, the frequency region indicated by B1 in FIG. 27, where the value of MTF decreases to 0. In this way, at higher frequencies, it is difficult to accurately estimate a significant difference by the effect of the correction processing (step S1030). Therefore, in such a frequency range, blur correction need not be performed in the blur adjustment processing (step S1032).

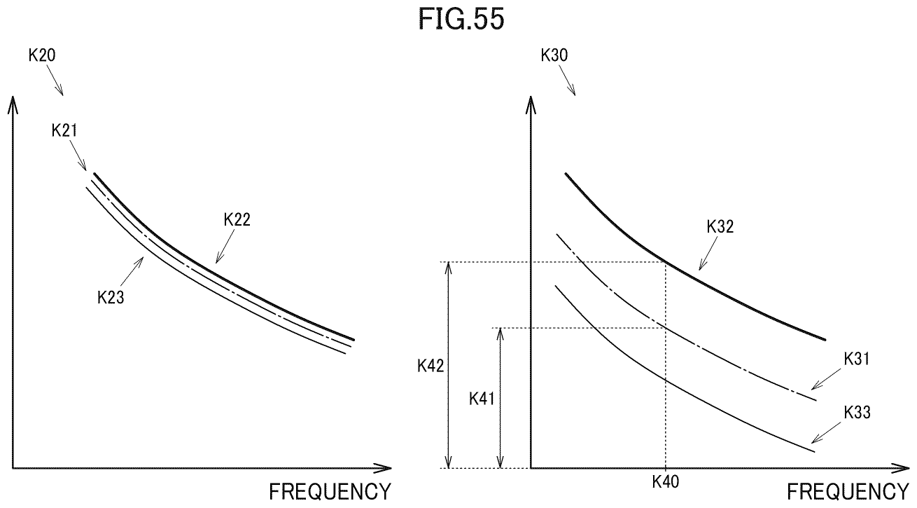

The frequency characteristic evaluation illustrated in FIG. 54 is performed for each object distance to produce, for example, a frequency characteristic diagram indicated by K20 or K30 in FIG. 55. The vertical axis is not specified, but may be contrast or amplitude. The graph of K20 illustrates a frequency characteristic when the object distance is the best focus distance. In the graph of K20, K21 is a first frequency characteristic, which is the frequency characteristic of the output image 134 obtained by the correction processing (step S1030). Further, K23 is a frequency characteristic of the training image 32 before the correction processing (step S1030) is performed.

K22 in the graph of K20 in FIG. 55 is a target frequency characteristic by the effect of the blur adjustment processing (step S1032), which is the frequency characteristic of the true image 36. More specifically, K22 is a plot of the result of frequency characteristic evaluation performed for an image generated by performing the best focus simulation processing (step S300) for the predetermined subject image 30 described above with reference to FIG. 10. Alternatively, K22 may be a plot of the result of frequency characteristic evaluation performed for the predetermined subject image 30 itself which is captured by the given imaging system 104 in focus as described above with reference to FIG. 11.

In the frequency characteristic diagram K20, since an image is captured at an object distance that is the best focus distance, the training image 32 is substantially the same as the true image 36, and the plots K21, K22, and K23 overlap each other. For convenience, in the graph K20, the plots K21, K22, and K23 are depicted slightly offset from each other.

In the frequency characteristic diagram K30, K31 is a first frequency characteristic which is the frequency characteristic of the output image 134 when an image is captured at an object distance other than the best focus distance, and K33 is a frequency characteristic of the training image 32. On the other hand, K32 is plotted by converting data of a second frequency characteristic indicated by K22 into the frequency characteristic diagram K30.

Then, for example, a value of the correction factor at a frequency indicated by K40 is determined, based on a value on the horizontal axis indicated by K41, a value on the horizontal axis indicated by K42, and the like, whereby the correction factor at a desired object distance and frequency, that is, one record in the correction table 1400 in FIG. 25, can be obtained. Therefore, the correction table 1400 can be created by repeating the same technique as many as the number of combinations of the number of object distance data and the number of frequency data as necessary.

Based on the foregoing, in the information processing system 1000 according to the present embodiment, the correction table 1400 is a table in which the filter characteristic in the blur adjustment processing for matching the first frequency characteristic which is the frequency characteristic of the output image 134 to the second frequency characteristic which is a target frequency characteristic in the blur adjustment processing is associated with each of a plurality of object distances. The frequency characteristic is a function that quantitatively represents the relationship of contrast or amplitude to frequency.

In this way, the blur adjustment processing (step S1032) can be performed using the correction table 1400 including appropriate correction factors. Specifically, the blur adjustment processing (step S1032) is performed for the output image 134 based on the correction table 1400 created in this way, and the frequency characteristic based on the resulting blur adjustment processed image 1034 is as indicated by K32. As described above, since the frequency characteristic indicated by K32 is a frequency characteristic based on the true image 36, the output image 134 can be corrected to an image equivalent to the true image 36 by further performing the blur adjustment processing (step S1032) for the output image 134.

Further, in the information processing system 1000 according to the present embodiment, the second frequency characteristic may be created by converting information of an image obtained by capturing an image of a subject in focus by the given imaging system, or an image generated by the best focus simulation processing, into a frequency characteristic at an object distance other than the object distance at which the given imaging system is focused. In this way, a target frequency characteristic can be set as appropriate at an object distance other than the object distance that achieves focus. As a result, values of the correction factors in the correction table 1400 can be set as appropriate.

Further, in the information processing system 1000 according to the present embodiment, the training image 32 may be an image of biological tissue or an image of a subject that imitates biological tissue. In this way, the frequency characteristic can be evaluated using a subject with a contrast close to that of an actual subject to be imaged. As a result, blur correction can be performed appropriately for a processing target image with a contrast close to that of an actual subject to be imaged.

Further, in the correction table 1400 in the information processing system 1000 according to the present embodiment, the filter characteristic for blur correction associated with an object distance at which the MTF is zero at a frequency equal to or lower than the Nyquist frequency may be a filter characteristic that does not correct a blur for a frequency equal to or higher than a frequency at which the MTF is zero. In this way, appropriate blur correction processing can be performed for an image having frequency components in which a blur correction effect is obvious, among processing target images.

In the above example, the correction processing (step S1030) is performed for the whole of one processing target image. However, the technique of the present embodiment is not limited to this. For example, the processing target image may be divided and the correction processing (step S1030) may be performed for each of the divided processing target images.

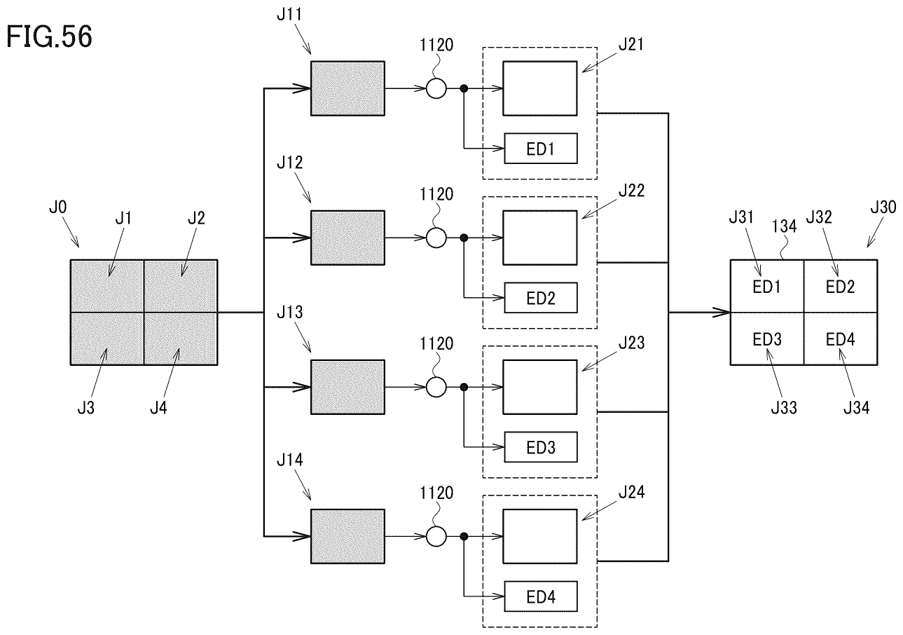

Specifically, for example, it is assumed that a processing target image indicated by J0 in FIG. 56 is input to the input section 1140 of the information processing system 1000. In the correction processing (step S1030), the processing section 1130 performs processing of dividing the processing target image indicated by J0 into four images, namely, an image indicated by J11, an image indicated by J12, an image indicated by J13, and an image indicated by J14. The image indicated by J11 corresponds to a region indicated by J1 in the original processing target image. The image indicated by J12 corresponds to a region indicated by J2 in the original processing target image. The image indicated by J13 corresponds to a region indicated by J3 in the original processing target image. The image indicated by J14 corresponds to a region indicated by J4 in the original processing target image. Although FIG. 56 illustrates an example in which a processing target image is divided into four images, the number of divided images is not limited to four, but can be determined by a user as appropriate.

The processing section 1130 then performs correction processing (step S1030) for the images indicated by J11, J12, J13, and J14 in sequence. In other words, the processing section 1130 reads the trained model 1120 from the memory section 1110 and inputs the image indicated by J11 to the trained model 1120. As a result, an image indicated by J21 and the estimated object distance label 1070 indicating an estimated object distance ED1 are output from the trained model 1120. Similarly, the processing section 1130 reads the trained model 1120 from the memory section 1110 and inputs the image indicated by J12 to the trained model 1120. As a result, an image indicated by J22 and the estimated object distance label 1070 indicating an estimated object distance ED2 are output from the trained model 1120. Similarly, the processing section 1130 reads the trained model 1120 from the memory section 1110 and inputs the image indicated by J13 to the trained model 1120. As a result, an image indicated by J23 and the estimated object distance label 1070 indicating an estimated object distance ED3 are output from the trained model 1120. Similarly, the processing section 1130 reads the trained model 1120 from the memory section 1110 and inputs the image indicated by J14 to the trained model 1120. As a result, an image indicated by J24 and the estimated object distance label 1070 indicating an estimated object distance ED4 are output from the trained model 1120.

The images indicated by J21, J22, J23, and J24 can be regarded as divided images of the output image 134 obtained by performing the correction processing (step S1030) for the subject image indicated by J0. The processing section 1130 then performs processing of combining the images indicated J21, J22, J23, and J24 into the output image 134 indicated by J30. A region indicated by J31 in the output image 134 indicated by J30 corresponds to the image indicated by J21. Similarly, a region indicated by J32 in the output image 134 indicated by J30 corresponds to the image indicated by J22, a region indicated by J33 corresponds to the image indicated by J23, and a region indicated by J34 corresponds to the image indicated by J24.

Further, for example, information on the estimated object distance ED1 corresponding to the image J21 may be superimposed and displayed on the region indicated by J31. Similarly, information on the estimated object distance ED2 corresponding to the image J22 may be superimposed and displayed on the region indicated by J32, information on the estimated object distance ED3 corresponding to the image J23 may be superimposed and displayed on the region indicated by J33, and information on the estimated object distance ED4 corresponding to the image J24 may be superimposed and displayed on the region indicated by J34.

In addition to the correction processing (step S1030), the blur adjustment processing (step S1032) may be further performed for each of the divided processing target images. In FIG. 57, the processing section 1130 performs processing of dividing the processing target image indicated by J0 in the correction processing (step S1030) into the images indicated by J11, J12, J13, and J14, in the same manner as in FIG. 56. The processing section 1130 then performs the correction processing (step S1030) for each of the images indicated by J11, J12, J13, and J14.

Based on the foregoing, in the information processing system 100 according to the present embodiment, the processing section 1130 estimates an object distance for each predetermined divided region of a processing target image, acquires from the correction table 1400 a filter characteristic associated with the object distance estimated for each divided region, and performs blur adjustment processing for the output image 134 using the acquired filter characteristic. In this way, the blur adjustment processing can be performed according to the object distance estimated for each divided region. As a result, the blur adjustment processing can be performed in consideration of the flatness of a subject.

The processing in step S1034 in FIG. 24 will be described more specifically. FIG. 57 is a conceptual illustration of the processing in step S1034. In FIG. 57, K50 is a conceptually simplified illustration of the filter characteristic as a real-space filter, which is inverse Fourier transform of the frequency response function indicated by H12 in FIG. 25. In other words, step S1034 includes processing of performing inverse Fourier transform of the frequency response function and processing of performing convolution of a real-space filter obtained by the inverse Fourier transform for the output image 134. Since the image obtained by convolution corresponds to the blur adjustment processed image 1034, the processing section 1130 outputs the blur adjustment processed image 1034 as a corrected image from the output section 1150 in step S1040 in FIG. 18. In this way, in the information processing system 1000 according to the present embodiment, the filter characteristic is a real-space filter, and the processing section 1130 performs blur adjustment processing by performing convolution of the real-space filter for the output image 134. In this way, the blur adjustment processing in real space can be performed.

Further, the processing section 1130 may perform step S1034 in FIG. 24 in frequency space. FIG. 58 is a conceptual illustration of the processing in step S1034 performed in frequency space. In FIG. 58, K60 is a conceptually simplified illustration of a frequency signal obtained by Fourier transform of the output image 134. Further, K61 in FIG. 58 is a conceptually simplified illustration of a frequency signal obtained by multiplying the frequency signal of K60 by the frequency response function indicated by H12 in FIG. 25. In other words, step S1034 includes processing of performing Fourier transform of the output image 134, processing of multiplying a frequency signal obtained by the Fourier transform by the frequency response function as the filter characteristic obtained in step S1033, and processing of performing inverse Fourier transform of a frequency signal obtained by the multiplication. Since the image obtained by inverse Fourier transform corresponds to the blur adjustment processed image 1034, the processing section 1130 outputs the blur adjustment processed image 1034 as a corrected image from the output section 1150 in step S1040 in FIG. 18.

Based on the foregoing, in the information processing system 1000 according to the present embodiment, the filter characteristic is a frequency response function, and the processing section 1130 performs blur adjustment processing by performing Fourier transform of the output image 134, multiplying a frequency signal acquired by the Fourier transform by the frequency response function, and performing inverse Fourier transform of a frequency signal acquired by the multiplication. In this way, the blur adjustment processing in frequency space can be performed.

The technique in FIG. 57 and the technique in FIG. 58 are mathematically equivalent. Therefore, the blur adjustment processed 1034 obtained by the technique in FIG. 57 is substantially the same as the blur adjustment processed image 1034 obtained by the technique in FIG. 58. Whether to perform step S1034 by the technique in FIG. 57 or to perform step S1034 by the technique in FIG. 58 can be determined as appropriate by a user.

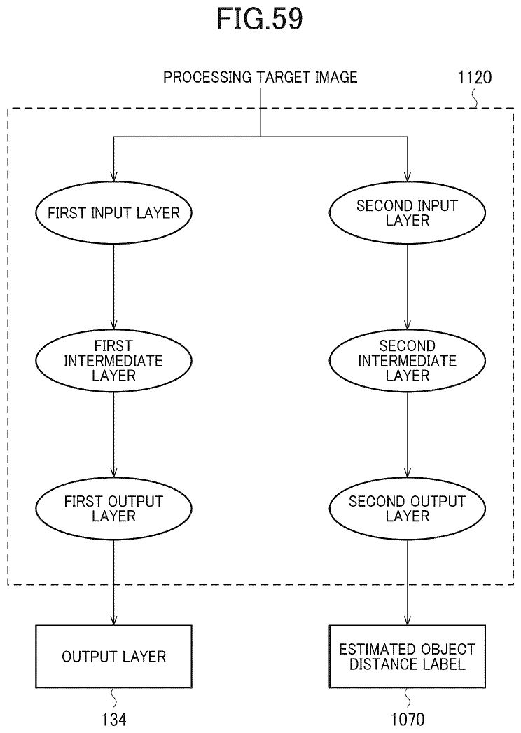

For example, the trained model 1120 according to the present embodiment may be constructed with two or more neural networks. Specifically, for example, as illustrated in FIG. 59, the trained model 1120 may include a first input layer, a second input layer, a first intermediate layer, a second intermediate layer, a first output layer, and a second output layer. For example, a processing target image is input to the first input layer. The first intermediate layer performs computation for an output from the first input layer. The first output layer then generates the output image 134 from an output from the first intermediate layer. Further, the processing target image is input to the second input layer. The second intermediate layer performs computation for an output from the second input layer. The second output layer then generates the estimated object distance label 1070 from an output from the second intermediate layer. Whether to construct the trained model 1120 as illustrated in FIG. 23 or as illustrated in FIG. 59 can be determined as appropriate by a user.

Although the embodiments have been described in detail above, it will be readily understood by those skilled in the art that many modifications can be made without substantially departing from the novel matters and effects in the present disclosure. Therefore, all such modifications are intended to be included within the scope of the present disclosure. Any term cited with a different term having a broader meaning or the same meaning at least once in the specification and the drawings can be replaced by the different term in any place in the specification and the drawings. All combinations of the embodiments and modifications are also included in the scope of the present disclosure. The configuration, operation, and the like of the information processing system, the endoscope system, the image processing method, and the program are not limited to those described in the embodiments and can be modified in various ways.

1. An information processing system comprising:
a memory configured to store a trained model trained by machine learning with a data set including a training image, a true image, and an object distance label, and a correction table in which an object distance is associated with a filter characteristic for blur correction, the object distance being a distance between an imaging system and a subject; and
one or more processors, wherein
the training image is generated by performing defocus simulation processing that simulates, for a predetermined subject image in focus captured by a given imaging system, an effect of a blur caused by defocus of a first imaging system, based on a transfer function or a point spread function of the first imaging system at a predetermined object distance,
the defocus simulation processing is performed for a region on an optical axis of the first imaging system and a region other than on the optical axis in the training image, based on the transfer function or the point spread function on the optical axis,
the true image is an image generated by performing best focus simulation processing that simulates, for the predetermined subject image, a state in which the first imaging system is focused, based on the transfer function or the point spread function at the object distance at which the first imaging system is focused, or the predetermined subject image itself,
the trained model is trained by machine learning so that the training image is the true image, and trained by machine learning by applying, as the object distance label, the object distance of the transfer function or the point spread function of the first imaging system used in the defocus simulation processing, and
the one or more processors
use the trained model to generate an output image in which a blur of a processing target image is corrected, the processing target image being an image captured by the first imaging system,
estimate the object distance of the processing target image, and
acquire, from the correction table, the filter characteristic associated with the estimated object distance, and perform blur adjustment processing for the output image using the acquired filter characteristic.

2. The information processing system according to claim 1, wherein
the correction table is a table in which the filter characteristic in the blur adjustment processing for matching a first frequency characteristic to a second frequency characteristic is associated with each of a plurality of the object distances, the first frequency characteristic being a frequency characteristic of the output image, the second frequency characteristic being a target frequency characteristic in the blur adjustment processing, and
the frequency characteristic is a function that quantitatively represents a relationship of contrast or amplitude to frequency.

3. The information processing system according to claim 2, wherein the second frequency characteristic is created by converting information of an image obtained by capturing an image of a subject in focus by the given imaging system, or an image generated by the best focus simulation processing, into the frequency characteristic at the object distance other than the object distance at which the given imaging system is focused.

4. The information processing system according to claim 2, wherein
the filter characteristic is a frequency response function, and
the one or more processors perform the blur adjustment processing by performing Fourier transform for the output image, multiplying a frequency signal acquired by the Fourier transform by the frequency response function, and performing inverse Fourier transform of a frequency signal acquired by the multiplication.

5. The information processing system according to claim 2, wherein
the filter characteristic is a real-space filter, and
the one or more processors perform the blur adjustment processing by performing convolution of the real-space filter for the output image.

6. The information processing system according to claim 1, wherein
the trained model is constructed with one neural network,
the neural network includes
an input layer to which the processing target image is input,
an intermediate layer configured to perform computation for an output from the input layer,
a first output layer configured to generate the output image from an output from the intermediate layer, and
a second output layer configured to estimate the object distance from an output from the intermediate layer.

7. The information processing system according to claim 1, wherein
the one or more processors
estimate the object distance for each of predetermined divided regions of the processing target image, and
acquire, from the correction table, the filter characteristic associated with the estimated object distance for each of the divided regions, and perform the blur adjustment processing for the output image using the acquired filter characteristic.

8. The information processing system according to claim 1, wherein the training image is an image of biological tissue, or an image of a subject that imitates the biological tissue.

9. The information processing system according to claim 1, wherein in the correction table, the filter characteristic for the blur correction associated with the object distance at which a modulation transfer function is zero at a frequency equal to or lower than Nyquist frequency is the filter characteristic that does not correct the blur for a frequency equal to or higher than a frequency at which the modulation transfer function is zero.

10. An endoscope system comprising:
the information processing system according to claim 1; and
an endoscopic scope configured to capture the processing target image.

11. An image processing method using a trained model trained by machine learning with a data set including a training image, a true image, and an object distance label, and a correction table in which an object distance is associated with a filter characteristic for blur correction, the object distance being a distance between an imaging system and a subject, wherein
the training image is generated by performing defocus simulation processing that simulates, for a predetermined subject image in focus captured by a given imaging system, an effect of a blur caused by defocus of a first imaging system, based on a transfer function or a point spread function of the first imaging system at a predetermined object distance,
the defocus simulation processing is performed for a region on an optical axis of the first imaging system and a region other than on the optical axis in the training image, based on the transfer function or the point spread function on the optical axis,
the true image is an image generated by performing best focus simulation processing that simulates, for the predetermined subject image, a state in which the first imaging system is focused, based on the transfer function or the point spread function at the object distance at which the first imaging system is focused, or the predetermined subject image itself,
the trained model is trained by machine learning so that the training image is the true image, and trained by machine learning by applying, as the object distance label, the object distance of the transfer function or the point spread function of the first imaging system used in the defocus simulation processing,
the image processing method comprising:
using the trained model to generate an output image in which a blur of a processing target image is corrected, the processing target image being an image captured by the first imaging system;
estimating the object distance of the processing target image; and
acquiring, from the correction table, the filter characteristic associated with the estimated object distance, and performing blur adjustment processing for the output image using the acquired filter characteristic.

12. A non-transitory information storage medium that stores a program for causing a computer to execute the image processing method according to claim 11.
